(19) United States
(12) Patent Application Publication
(10) Pub. No.: US 2015/0238334 A1
(43) Pub. Date: Aug. 27, 2015

(54) Implant for Treating Aneurysms

(71) Applicants: Kevin Kang, San Jose, CA (US); Jeffrey Madsen Pappas, Santa Clara, CA (US); David I. Levy, San Diego, CA (US)

(72) Inventors: Kevin Kang, San Jose, CA (US); Jeffrey Madsen Pappas, Santa Clara, CA (US); David I. Levy, San Diego, CA (US)

(21) Appl. No.: 14/264,795

(22) Filed: Apr. 29, 2014

Related U.S. Application Data
(60) Provisional application No. 61/967,128, filed on Mar. 6, 2014.
(60) Provisional application No. 61/966,804, filed on Mar. 4, 2014.
(60) Provisional application No. 61/966,483, filed on Feb. 23, 2014.

(51) Int. Cl. A61F 2/90

(52) U.S. Cl. CPC A61F 2/90; A61F 2230/0017; A61F 2230/0008; A61F 2002/823

(57) ABSTRACT

Implants and methods used for the treatment of aneurysms are disclosed. More particularly, embodiments of an implant having a circumferentially discontinuous aneurysm section for the treatment of cerebral aneurysms are disclosed.

This application claims the benefit of U.S. Provisional Patent Application Nos. 61/966,483 filed on Feb. 23, 2014, 61/966,804 filed on Mar. 4, 2014, and 61/967,128 filed on Mar. 6, 2014, and this application hereby incorporates herein by reference those provisional patent applications.

### BACKGROUND

1. Field

The present invention relates to implants used for the treatment of aneurysms. More particularly, embodiments of the present invention relate to an implant for treating cerebral aneurysms in tortuous, bifurcated and non-bifurcated, anatomies.

2. Background Information

Referring to FIG. 1, a pictorial view illustrating a patient with a cerebral aneurysm is shown. Aneurysms are pathological bulges in vascular anatomies, typically caused either by disease or weakening of a vessel wall. An aneurysm 100 may occur in the cerebral vessels 102 of a patient 104, such as in the vertebral, basilar, middle cerebral, posterior cerebral, or internal carotid arteries. Typically, cerebral vessels include vessel diameters in a range of between about 1.5 to 5.5 mm (3.5 mm average).

Referring to FIG. 2, a detail view, taken from Detail A of FIG. 1, of a stent deployed at an aneurysm site is shown. Cerebral aneurysm 100 may be classified as a saccular aneurysm, having an aneurysm sac 202 joined with a portion of a vessel 102 at an aneurysm gate. Unless an aneurysm is depressurized, the aneurysm may eventually rupture, leading to severe complications. For example, in the case of cerebral aneurysms, a ruptured aneurysm may lead to severe intracranial hemorrhage with associated loss of perception, loss of balance, or even death.

Numerous approaches exist to treat cerebrovascular aneurysms, including some minimally invasive techniques. For example, an endovascular coiling procedure may be used in which a microcatheter is tracked to an aneurysm site and one or more embolic coils 204 is inserted into aneurysm sac 202 to promote blood clotting, which occludes and depressurizes the sac.

Placing a stent 206 across the aneurysm gate may be used as an adjunct to, or a replacement for, embolic coil 204. For example, in a technique referred to as “jailing”, stent 206 may be delivered to scaffold the aneurysm gate and to create and/or retain a thrombus within the aneurysm sac. Thus, by jailing embolic coil 204, aneurysm sac 202 may be occluded and depressurized. Stent may be deployed across the aneurysm gate before or after inserting embolic coil 204 into aneurysm sac 202. In an alternative embodiment, stent 206 may be used alone, without also deploying embolic coil 204. In such cases, stent 206 may act as a flow diverter that slows or prevents blood flow into the aneurysm sac 202 with the goal of removing flow and depressurizing sac 202.

Referring to FIG. 3, a cross-sectional view of a stent deployed at an aneurysm site is shown. Aneurysms may occur in various anatomies. For example, vessel 102 may be highly tortuous. An illustration of such tortuous anatomy is represented in FIG. 2, showing a substantial curvature of vessel 102 adjacent the aneurysm gate of aneurysm 100. A cross-sectional view of vessel 102 illustrates that a tortuous vessel may nonetheless exhibit a circular cross-sectional profile. However, typical stents, particularly stents formed from self-expandable materials, may exhibit a tendency to “pancake” when they are expanded within the tortuous anatomy. For example, stent 206 may kink due to the stent structure being over-constrained and/or too stiff to conform to vessel 102 profile. Thus, stent 206 may become “ovalized”. As a consequence of stent ovally deforming, a space 302 may be formed between an outer diameter of stent 206 and a wall of vessel 102. Space 302 may provide a location for emboli to form, which can lead to negative medical outcomes.

Referring to FIG. 4, a pictorial view of an attempt to pass a delivery system through a stent deployed at a site of a bifurcation aneurysm is shown. Aneurysm 100 may be located at a bifurcation ostium, e.g., between main vessel 102 and a branch vessel 402. In such cases, it may be necessary to use two stents to jail aneurysm 100. For example, a first stent 206 may be used to jail a rightmost side of aneurysm 100, and a second stent may be used to jail a leftmost side of aneurysm 100. However, such a technique may require passing guidewire 404 and delivery system 406 through first stent 206 in the main vessel 102 to allow placement of the second stent in the branch vessel 402. As shown, crossing through stent 206 with guidewire 404 and/or delivery system 406 may be difficult since guidewire 404 or delivery system 406 may snag on the stent pattern. This can lead to stent damage and associated negative medical outcomes. Such stent delivery challenges may be exacerbated when stent 206 includes a closed cell stent pattern since the cells will not yield to the catheter and snagging is therefore more likely.

Despite the challenge of crossing closed cell patterns with secondary devices, treating aneurysms with closed cell stent patterns may be medically desirable, since closed cell stents can allow for stent retrieval, and thus, may allow for control of the positioning of the implant, e.g., rotational and translational movement of the implant, during placement in the anatomy. That is, a closed-cell self-expandable stent may be partially deployed from a stent delivery system, and then be retrieved back into the delivery system if repositioning is required. Thus, a retrievable stent design may be a valuable feature during treatment of aneurysms, because it may allow for repeated attempts at placement to ensure that aneurysm 100 is properly jailed. Accordingly, aneurysm treatment may benefit from an implant that conforms fully to a target vessel, that does not resist passage of a secondary device through the implant into a branch vessel, and that is fully retrievable to facilitate accurate placement relative to an aneurysm.

### SUMMARY OF THE DESCRIPTION

Implants used for treating aneurysms are disclosed. In an embodiment, a vascular implant is provided having an unexpanded state and an expanded state. The vascular implant may include a proximal transition ring having a plurality of ring undulations contiguous about a longitudinal axis such that the proximal transition ring is continuous around a circumference of the vascular implant. The vascular implant may also include a plurality of aneurysm section holders extending longitudinally from respective ring undulations to a distal transition ring. In an embodiment, the plurality of aneurysm section holders are further separated by a void. An aneurysm arc having a plurality of arc undulations contiguous about the longitudinal axis may be opposite of the plurality of aneurysm section holders from the void such that the aneurysm arc is discontinuous and extends substantially around the circumference in the unexpanded state and the expanded state.

In an embodiment, a vascular implant includes ring undulations that are contiguously coupled with each other by a plurality of transition joints and arc undulations that are contiguously coupled with at least one of each other or an aneurysm section holder by a plurality of arc joints. Furthermore, a plurality of transition connectors may couple the plurality of transition joints with the plurality of arc joints. The vascular implant may further include a same number of transition joints in a proximal transition ring as arc joints in an aneurysm arc.

In an embodiment, a vascular implant includes a transition ring with a constraint undulation and a plurality of transition undulations. A plurality of aneurysm section holders may extend longitudinally from the constraint undulation to a distal transition ring. Furthermore, the constraint undulation may include a first composite stiffness, and each of the plurality of transition undulations may include a respective second composite stiffness. The first composite stiffness may be greater than each of the respective second composite stiffness. Accordingly, the constraint undulation may include a plurality of constraint struts having a first length and the expansion undulations may include a plurality of expansion struts having a second length, and the first length may be shorter than the second length. Alternatively, the constraint undulation may include a constraint joint having a first width and the plurality of expansion undulations may include a plurality of transition joints having a second width, and the first width may be greater than the second width.

In an embodiment, a vascular implant may include a proximal transition ring having a stack undulation. The stack undulation may include a plurality of stack struts interconnected by a stack joint, and the stack struts may include respective ends coupled with respective constraint struts of a constraint undulation.

In an embodiment, a vascular implant may include a void with a width along a longitudinal length of an arc segment. The width may have a distance less than about ten percent of the circumference of the vascular implant in an unexpanded state and an expanded state. For example, in an embodiment, the width has a distance in a range of about one percent to seven percent—of the circumference of the vascular implant in the unexpanded state and the expanded state. The void may also include a port between a proximal transition ring and a distal transition ring of the vascular implant, and the port may include a shape configured to allow a catheter to pass through the void from an inner lumen of the vascular implant. For example, the shape may include a projected area selected from a group consisting of an ellipse and a polygon. Furthermore, a geometric surface defined by the void may be configured to remain substantially the same when the vascular implant expands from the unexpanded state to the expanded state. Alternatively, the geometric surface defined by the void may be configured to increase when the vascular implant expands from the unexpanded state to the expanded state.

In an embodiment, a vascular implant may include aneurysm section holders that extend substantially straightly in an axial direction from respective ring undulations to a distal transition ring. One or more aneurysm marker holder may be located adjacent to the plurality of aneurysm section holders, and an aneurysm marker may be in each aneurysm marker holder. The one or more aneurysm marker holder may be circumferentially between at least one of the plurality of aneurysm section holders or a plurality of constraint struts of a constraint undulation of the ring undulation. In an embodiment, a vascular implant may include a radial connector interconnecting a plurality of aneurysm section holders at an intermediate location between a proximal transition ring and a distal transition ring. The radial connector may include an intermediate aneurysm marker holder and an intermediate aneurysm marker may be placed in the intermediate aneurysm marker holder. In an embodiment, a first aneurysm marker holder may be adjacent to a first aneurysm section holder and a second aneurysm marker holder may be adjacent to a second aneurysm section holder. Furthermore, a first aneurysm marker in the first aneurysm marker holder may be shaped differently than a second aneurysm marker in the second aneurysm marker holder.

In an embodiment, a vascular implant may include a first end marker holder opposite of a proximal transition ring from an aneurysm arc, and a second end marker holder opposite of a distal transition ring from an aneurysm arc. Furthermore, an end marker may be placed in each end marker holder.

In an embodiment, a vascular implant includes an aneurysm arc with a first arc pattern circumferentially between a first of a plurality of aneurysm section holders and a plane, and a second arc pattern circumferentially between a second of the plurality of aneurysm section holders and the plane. The plane may pass through a void and a longitudinal axis of the vascular implant. Furthermore, the first arc pattern and the second arc pattern may be symmetric with each other over at least a portion of their respective arc lengths.

### BRIEF DESCRIPTION OF THE DRAWINGS

FIG. 1 is a pictorial view illustrating a patient with a cerebral aneurysm.

FIG. 2 is a detail view, taken from Detail A of FIG. 1, of a stent deployed at an aneurysm site.

FIG. 3 is a cross-sectional view of a stent deployed at an aneurysm site.

FIG. 4 is a pictorial view of an attempt to pass a delivery system through a stent deployed at a site of a bifurcation aneurysm.

FIG. 5 is a perspective view of a vascular implant in an unexpanded state in accordance with an embodiment of the invention.

FIG. 6 is a perspective view of a vascular implant in an expanded state in accordance with an embodiment of the invention.

FIG. 7 is a flat pattern illustration of a vascular implant having a void that enlarges during expansion in accordance with an embodiment of the invention.

FIG. 8 is a side view of a constraint undulation region of a vascular implant in accordance with an embodiment of the invention.

FIG. 9 is a perspective view of a vascular implant in an unexpanded state in accordance with an embodiment of the invention.

FIG. 10 is a perspective view of a vascular implant in an expanded state in accordance with an embodiment of the invention.

FIG. 11 is a flat pattern illustration of a vascular implant in accordance with an embodiment of the invention.

FIG. 12 is a side view of a distal constraint undulation region of a vascular implant in accordance with an embodiment of the invention.

FIG. 13 is a side view of a proximal constraint undulation region of a vascular implant in accordance with an embodiment of the invention.

FIG. 14 is a side view of a constraint undulation region of a vascular implant in accordance with an embodiment of the invention.

FIG. 15 is a side view of a constraint terminal region of a vascular implant in accordance with an embodiment of the invention.

FIG. 16 is a perspective view of a vascular implant in an unexpanded state in accordance with an embodiment of the invention.

FIG. 17 is a perspective view of a vascular implant in an expanded state in accordance with an embodiment of the invention.

FIG. 18 is a flat pattern illustration of a vascular implant in accordance with an embodiment of the invention.

FIG. 19 is a pictorial view of a void configuration in accordance with an embodiment of the invention.

FIG. 20 is a pictorial view of a void configuration in accordance with an embodiment of the invention.

FIG. 21 is a pictorial view of a void configuration in accordance with an embodiment of the invention.

FIG. 22 is a flat pattern illustration of a slanted aneurysm section pattern of a vascular implant in accordance with an embodiment of the invention.

FIG. 23 is a flat pattern illustration of an hourglass aneurysm section pattern of a vascular implant in accordance with an embodiment of the invention.

FIG. 24 is a pictorial view of a vascular implant deployed at an aneurysm site in accordance with an embodiment of the invention.

FIG. 25 is a cross-sectional view of a vascular implant deployed at an aneurysm site in accordance with an embodiment of the invention.

FIG. 26 is a pictorial view of a delivery system passing through a vascular implant deployed at a site of a bifurcation aneurysm in accordance with an embodiment of the invention.

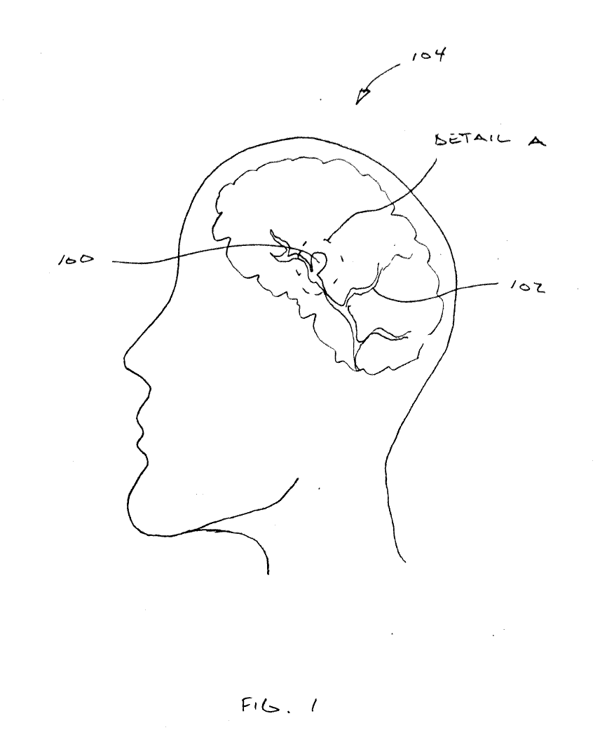

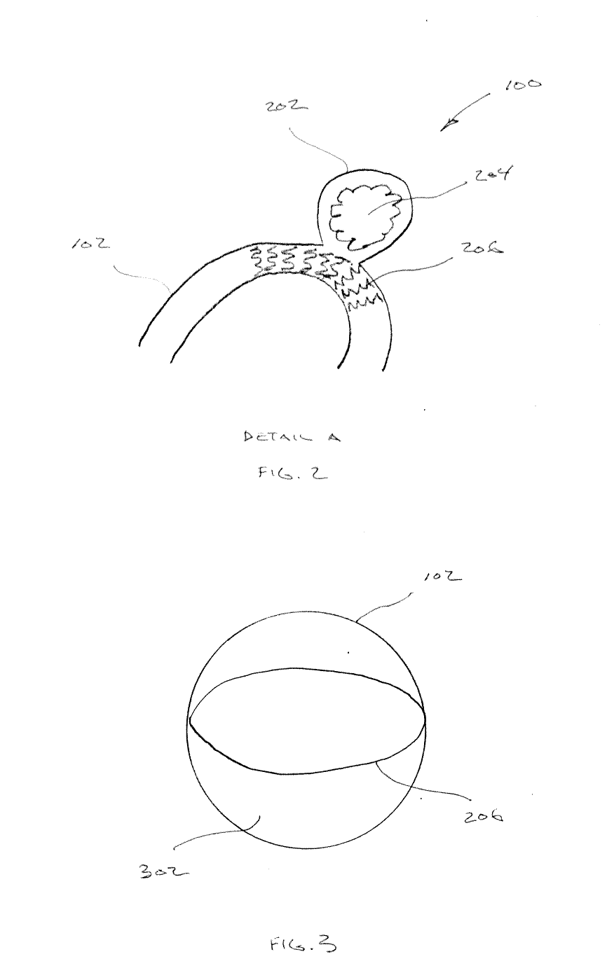

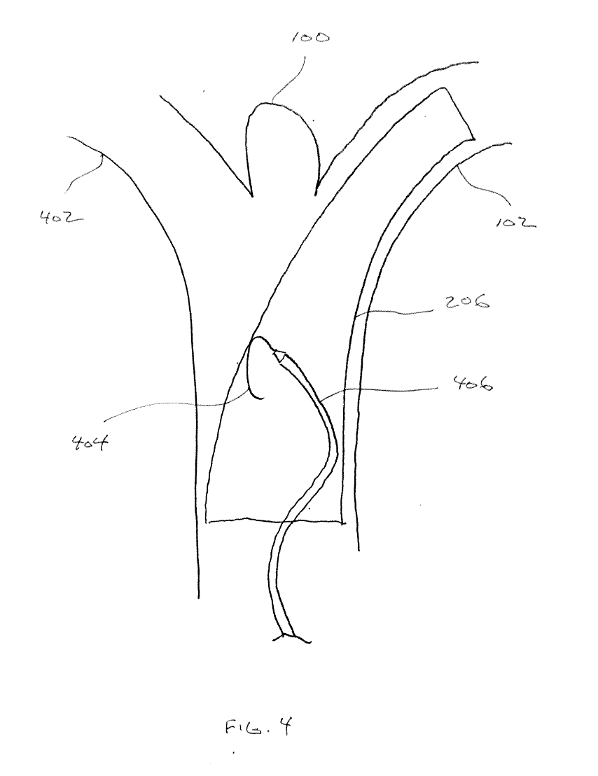

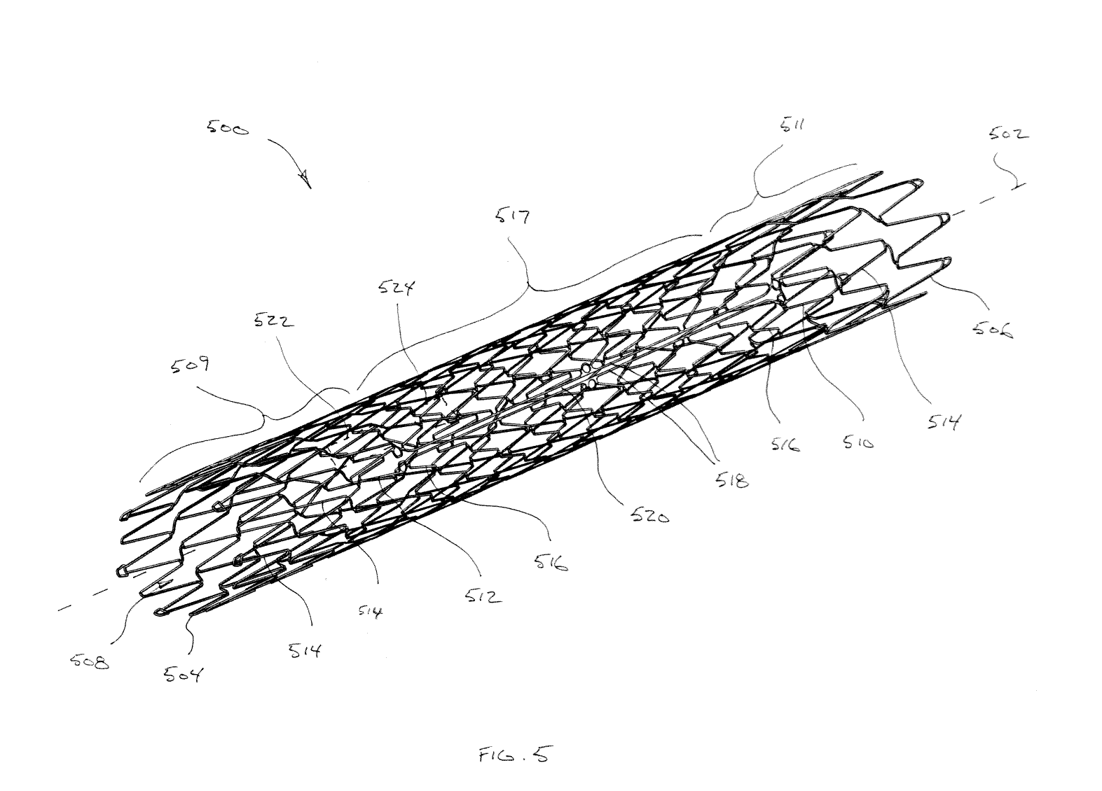

### DETAILED DESCRIPTION OF THE INVENTION

While some embodiments of the present invention are described with specific regard to neurovascular applications, the embodiments of the invention are not so limited and certain embodiments may also be applicable to the treatment of aneurysms in other body vessels. For example, embodiments of the invention may be used to treat aneurysms distal to the origin of the renal arteries, thoracic aortic aneurysms, popliteal vessel aneurysms, or any other body vessel locations.

In various embodiments, description is made with reference to the figures. However, certain embodiments may be practiced without one or more of these specific details, or in combination with other known methods and configurations. In the following description, numerous specific details are set forth, such as specific configurations, dimensions, and processes, in order to provide a thorough understanding of the present invention. In other instances, well-known processes and manufacturing techniques have not been described in particular detail in order to not unnecessarily obscure the present invention. Reference throughout this specification to “one embodiment,” “an embodiment”, or the like, means that a particular feature, structure, configuration, or characteristic described in connection with the embodiment is included in at least one embodiment of the invention. Thus, the appearances of the phrase “one embodiment,” “an embodiment”, or the like, in various places throughout this specification are not necessarily referring to the same embodiment of the invention. Furthermore, the particular features, structures, configurations, or characteristics may be combined in any suitable manner in one or more embodiments.

As described throughout this disclosure, the terms “substantially” and “generally” are used to indicate that the description approximates an actual configuration of an embodiment of the invention. For example, in a description that refers to an implant section as being “substantially cylindrical”, it is to be appreciated that the section may not extend fully around the circumference of the implant, but that one skilled in the art would recognize the section as extending almost entirely around the circumference in a cylindrical manner.

In an aspect, a vascular implant for treating aneurysms may include an aneurysm section having a plurality of aneurysm arcs opposite a void. The void may be positioned circumferentially opposite from an aneurysm during delivery, such that the aneurysm arcs may expand against the aneurysm gate to jail the aneurysm during expansion of the vascular implant. In an embodiment, the aneurysm section may be substantially cylindrical in both the unexpanded and the expanded state, but the void may nonetheless form a discontinuity between aneurysm arcs, such that the aneurysm section is discontinuous in a circumferential direction. The discontinuity of aneurysm section prevents over-constraint of aneurysm arcs such that vascular implant may be placed within a tortuous aneurysm site without pancaking or kinking. Instead, the discontinuous aneurysm section may wrap within itself to avoid ovally deforming and to conform to the surrounding vessel. Thus, the aneurysm section may appose aneurysm gate and jail an aneurysm without generating spaces that may lead to emboli formation.

In another aspect, a vascular implant having a discontinuous aneurysm section provides a port for a secondary device to be tracked through. For example, a void between aneurysm section may be sized to allow the aneurysm section remain substantially cylindrical in both an unexpanded state and an expanded state of the vascular implant. For example, in an embodiment, the void may remain less than about ten percent of the vascular implant circumference in both the unexpanded state and the expanded state. Thus, vascular implant may expand to scaffold a bifurcation aneurysm, but allow for a guidewire or stent delivery system to subsequently advance through the void into a branch vessel. Accordingly, a second implant may be deployed through the void, which may widen the void as needed and fully scaffold the bifurcation aneurysm.

In an aspect, a vascular implant having a discontinuous aneurysm section may be delivered to an aneurysm site accurately. The vascular implant may include a number of markers that indicate a location of a void between the aneurysm section, such that the void may be accurately oriented relative to the aneurysm during delivery and expansion of the vascular implant. Furthermore, the vascular implant may be fully retrievable into a delivery catheter, such that the vascular implant may be repeatedly advanced and retracted into the delivery catheter until the void orientation is repositioned as desired.

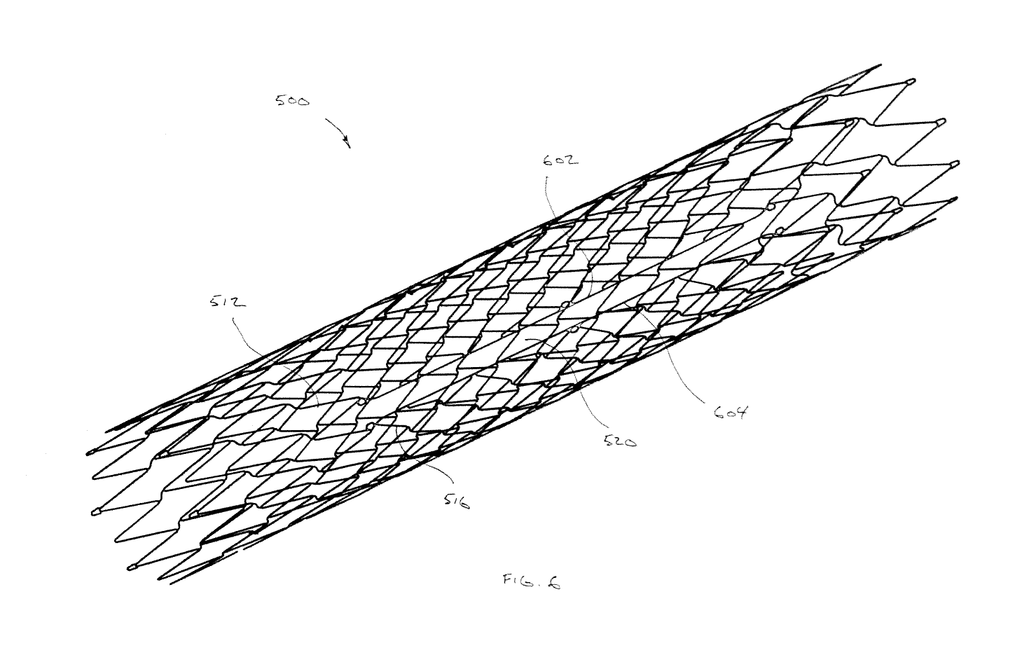

Referring to FIG. 5, a perspective view of a vascular implant in an unexpanded state is shown in accordance with an embodiment of the invention. In the unexpanded state, vascular implant 500 may include a generally cylindrical outer profile along a longitudinal axis 502 between a proximal end 504 and a distal end 506. Vascular implant 500 may have various scaffold structures extending in a circumferential direction around longitudinal axis 502 to form the cylindrical outer profile. For example, vascular implant 500 may have one or more rings and arcs arranged along longitudinal axis 502 that define a cylindrical outer profile and inner profile.

In an embodiment, some scaffold structures of vascular implant 500 extend continuously around a circumference of vascular implant 500. More particularly, vascular implant 500 may include one or more ring scaffold structures. By contrast, in an embodiment, some scaffold structures of vascular implant 500 do not extend continuously around the circumference of vascular implant 500, although they may extend nearly entirely around the circumference of vascular implant 500. More particularly, vascular implant 500 may include one or more arc scaffold structures with ends that are separated by a discontinuity.

The scaffold rings and scaffold arcs of vascular implant 500 may be differentiated by their respective locations in an overall structure of vascular implant 500. In an embodiment, vascular implant 500 includes one or more base rings 514 extending continuously around a circumference of vascular implant 500 in a proximal base section 509. Proximal base section 509 may extend from proximal end 504 to a proximal transition ring 512. Similarly, vascular implant 500 may include one or more base rings 514 extending continuously around a circumference of vascular implant 500 in a distal base section 511. Distal base section 511 may extend from distal end 506 to a distal transition ring 510. Proximal base section 509 and distal base section 511 may be configured to scaffold and restore blood flow in a parent vessel. More particularly, base sections may provide sufficient radial stiffness in an expanded state to anchor within the parent vessel and to resist inward loads applied by the vascular anatomy. Base rings 514 of base section may be configured accordingly.

In an embodiment, base rings 514 may include struts sized to flex and conform to a parent vessel wall, yet provide radial support for anchoring within the parent vessel. For example, in an embodiment, a width of base ring struts in a circumferential direction is in a range of about 0.001-inch to 0.005-inch. More particularly, the width may be in a range between about 0.001-inch to 0.003-inch. More particularly, in an embodiment, a width of base ring 514 struts may be about 0.0010-inch. A thickness of base ring 514 struts in a radial direction may be in a range of about 0.001-inch to 0.006-inch. More particularly, the thickness may be in a range between about 0.002-inch to 0.004 inch. For example, a thickness base ring 514 struts may be about 0.0024-inch.

In an embodiment, vascular implant 500 includes an aneurysm section 517 between proximal base section 509 and distal base section 511. Aneurysm section 517 may include one or more aneurysm arcs 516 that extend around a circumference of vascular implant 500, but which are not continuous around the entire circumference. For example, aneurysm arcs 516 may have a discontinuity that imparts to them an arc, as opposed to a ring, structure. Aneurysm section 517 may be configured to jail an aneurysm 100 in a parent vessel. More particularly, aneurysm section 517 may provide scaffolding substantially around vascular implant 500 to jail wide aneurysm gates. Aneurysm section 517 may be any length necessary to scaffold an anatomy of interest, e.g., approximately equal to an average aneurysm neck diameter. Aneurysms necks commonly have diameters of, e.g., about 5.5 mm. Thus, in an embodiment, aneurysm section 517 has an overall length of about 15 mm between transition rings 510, 512. Aneurysm arcs 516 may be configured accordingly.

In an embodiment, aneurysm arcs 516 may include struts sized to flex and conform to an aneurysm gate, yet be flexible enough so as to not over-constrain vascular implant 500 in a tortuous vessel. For example, in an embodiment, a width of aneurysm arc 516 struts in a circumferential direction is in a range of about 0.001-inch to 0.005-inch. More particularly, the width may be in a range between about 0.001-inch to 0.003-inch. More particularly, in an embodiment, a width of aneurysm arc 516 struts may be about 0.0010-inch. A thickness of aneurysm arc 516 struts in a radial direction may be in a range of about 0.001-inch to 0.006-inch. More particularly, the thickness may be in a range between about 0.002-inch to 0.004 inch. For example, a thickness aneurysm arc 516 struts may be about 0.0024-inch.

In an embodiment, aneurysm arcs 516 may be discontinuous with ends that connect with aneurysm section holders 518. Aneurysm section holders 518 may extend longitudinally between distal transition ring 510 and proximal transition ring 512. For example, aneurysm section holders 518 may extend straightly between the transition rings. Even more particularly, aneurysm section holders 518 may extend axially in the direction of longitudinal axis 502 over all or a portion of the distance between the transition rings. Aneurysm section holders 518 may be configured to support aneurysm section 517, but to provide sufficient flexibility such that aneurysm section holders 518 may move laterally to prevent oval deformation of the vascular implant 500.

Aneurysm section holders 518 may be sized and configured to provide adequate radial support to aneurysm section 517 yet be flexible enough to prevent over-constraining vascular implant 500, such as in the case where aneurysm section 517 is located in a tortuous vessel. For example, aneurysm section holders 518 may include a width, meaning a circumferential dimension, of between about 0.001-inch to 0.005-inch. More particularly, the width may be in a range between about 0.001-inch to 0.003-inch. More particularly, in an embodiment, a width of aneurysm section holders 518 may be about 0.0010-inch. A thickness of aneurysm section holders 518 in a radial direction may be in a range of about 0.001-inch to 0.006-inch. More particularly, the thickness may be in a range between about 0.002-inch to 0.004 inch. For example, a thickness of aneurysm section holders 518 may be about 0.0024-inch.

In an embodiment, aneurysm section holders 518 define a void 520. More particularly, void 520 may be located circumferentially between aneurysm section holders 518 and opposite of aneurysm section holders 518 from aneurysm section 517. Thus, void 520 may separate one aneurysm section holder 518 from another aneurysm section holder 518 over at least a portion of an axial length of aneurysm section 517, e.g., over an axial length of one or more of aneurysm arcs 516. Accordingly, void 520 may not only separate aneurysm section holders 518, but may also represent the circumferential discontinuity in one or more aneurysm arc 516. That is, aneurysm arcs 516 having ends that terminate at aneurysm section holders 518 may be contiguous over an arc length between aneurysm section holders 518, but the arc length may be less than a circumference of vascular implant 500 such that each aneurysm arc 516 is circumferentially discontinuous.

Void 520 may have a variety of shapes and sizes at least partially determined by aneurysm section holders 518. That is, since void 520 is defined between aneurysm section holders 518, void 520 varies as the path of each aneurysm section holder 518 varies. In an embodiment, aneurysm section holders 518 follow paths that are nearly identical, e.g., straight, curved, undulating, etc., between transition rings. Thus, a gap between aneurysm section holders may have a same distance over a portion of, or an entire length, of an aneurysm section holder 518. Accordingly, void 520 may have a same width over its entire length. Examples of a several other possible shapes and sizes of void 520 are discussed below.

In an embodiment, transition rings, such as distal transition ring 510 and proximal transition ring 512, may adapt respective base rings 514 to aneurysm section 517. For example, distal transition ring 510 may interconnect a base ring 514 of proximal base section 509 to a proximal aneurysm arc 516 of aneurysm section 517. Similarly, proximal transition ring 512 may interconnect a base ring 514 of distal base section 511 to a distal aneurysm arc 516 of aneurysm section 517. Transition rings may be configured to determine in part a degree to which aneurysm arcs 516 expand as vascular implant 500 transitions from an unexpanded state to an expanded state. Furthermore, transition rings may be configured to determine in part a degree to which aneurysm section holders 518 expand as vascular implant 500 transitions from an unexpanded state to an expanded state. Transition rings 510, 512 may be configured accordingly.

In an embodiment, transition rings 510, 512 may include struts sized to flex and conform to a parent vessel wall, yet provide radial support for anchoring within the parent vessel. Accordingly, at least a portion of transition ring struts may be sized similar to base ring 514 struts. For example, in an embodiment, a width of transition ring struts in a circumferential direction is in a range of about 0.001-inch to 0.005-inch. More particularly, the width may be in a range between about 0.001-inch to 0.003-inch. More particularly, in an embodiment, a width of transition ring struts may be about 0.0010-inch. A thickness of transition ring struts in a radial direction may be in a range of about 0.001-inch to 0.006-inch. More particularly, the thickness may be in a range between about 0.002-inch to 0.004 inch. For example, a thickness of transition ring struts may be about 0.0024-inch. In an embodiment, transition ring strut lengths and widths may vary around the circumference. For example, transition ring may include “transition undulations” and “constraint undulations”, each with different purposes and therefore different geometries and sizes.

Each ring or arc scaffold structure of vascular implant 500 may further include sub-elements generically referred to as “undulations”. An undulation may be any contiguous length of a ring or arc, such as a contiguous length of distal transition ring 510 or aneurysm arc 516. For example, a ring undulation 522 may be selected as a contiguous length of proximal transition ring 512 between two adjacent joints. Similarly, arc undulation 524 may be selected as a contiguous length of aneurysm arc 516 between two adjacent joints. Accordingly, each undulation may include at least two struts joined at a single peak between adjacent joints. Undulation geometries will be familiar to one skilled in the art and may include, for example, any combination of U-shaped, V-shaped, W-shaped, or other strut geometries extending between two adjacent joints that are generally aligned in a circumferential direction. Joints, which are described further below, may alternatively be referred to as “crowns”, “peaks”, “elbows”, “knees”, etc. Undulations may vary within a same scaffold structure and/or between different scaffold structures. For example, a first ring undulation 522 may have an axial length different from an adjacent ring undulation 522. Similarly, either of the first ring undulation 522 or the adjacent ring undulation 522 may have a same or different axial length from an arc undulation 524. Thus, the range of possible combinations of undulation geometries in vascular implant 500 is virtually unlimited. Nonetheless, the numerous embodiments provided below illustrate how such combinations may be made to provide vascular implant 500 within the scope of this description.

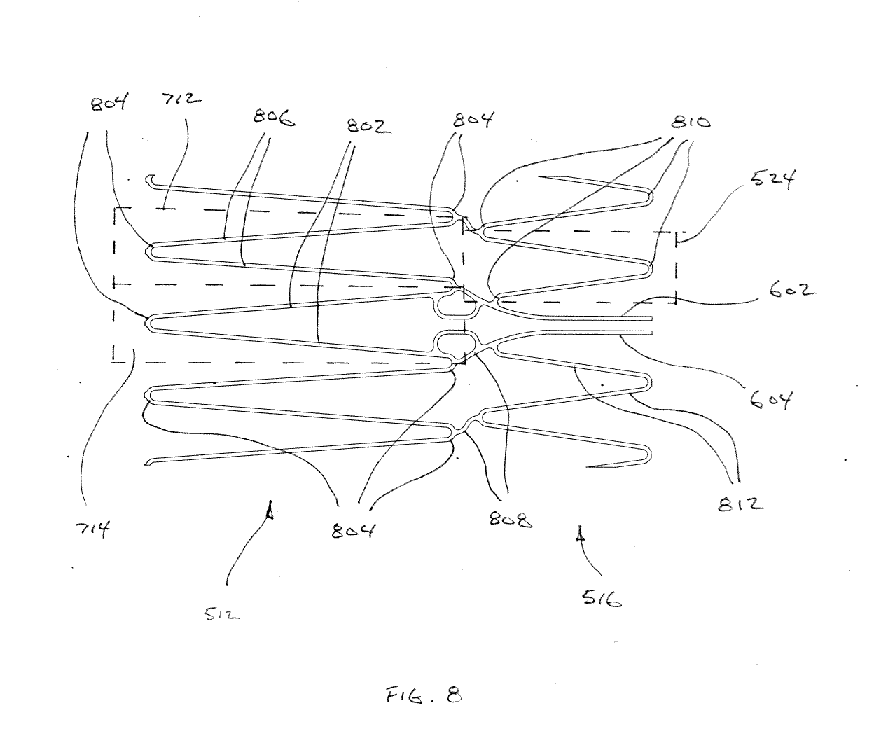

In an embodiment, a number of crowns in each scaffold structure may be varied. For example, base rings 514, transition rings 510, 512, and aneurysm arcs 516 may include the same number of joints. For example, a base ring 514, a proximal-most transition ring 512, and an aneurysm arc 516 may all have 14 crowns along an end at which they connect with each other. In an embodiment, a base ring 514, a distal-most transition ring 510, and an aneurysm arc 516 may not have the same number of joints. For example, the aneurysm arc 516 and the distal-most transition ring 510 may both include 14 crowns along an end at which they connect with each other, but the distal-most transition ring 510 may have a different number of crowns than an adjacent base ring 514 along an end at which they connect with each other, e.g., the rings may have 14 crowns and 9 crowns, respectively. In an alternative embodiment, a proximal-most transition ring 512 may have 17 crowns connected with 15 crowns of an adjacent aneurysm arc 516. Furthermore, an aneurysm arc 516 near a middle of aneurysm section 517 may include about 12 crowns, compared to 15 crowns of aneurysm arcs 516 near distal-most and proximal-most transition rings 510, 512. Thus, the number of crowns in each scaffold structure making up vascular implant 500 may vary, both between respective ends of the individual scaffold structures and between the different scaffold structures. Accordingly, some scaffold structures may have equal numbers of joints and others may not. Several embodiments representing scaffold structures with equal and different numbers of joints are illustrated in the accompanying figures, e.g., FIGS. 7, 11, and 18, which are described in further detail below.

The number of crowns in adjacent scaffold structures may also correlate with the number of connectors holding adjacent scaffold structures together. For example, as described further below, connectors may hold rings together, as well as hold rings together with arcs. Where adjacent rings or arcs have equal numbers of crowns, a connector may be placed between each crown to form a closed-cell pattern. Alternatively, when not every crown of a first scaffold structure is connected with an adjacent crown of an adjacent scaffold structure, an open-cell pattern may be formed. Closed-cell patterns are generally more retrievable than open-celled patterns, meaning that when every crown in vascular implant 500 is connected with an adjacent crown, vascular implant 500 may be fully retrievable into a delivery catheter with less risk of snagging a crown on the delivery catheter and damaging vascular implant 500. However, an open-cell design may also be made to be retrievable. For example, when one or more distal crown of a ring or arc is not linked with a proximal crown of an adjacent ring or arc, the distal crown may nonetheless be retrievable since the insertion of the distal crown into the delivery system may be through retrieval in a proximal direction, and thus, there may be no snag point on the distal crown relative to the catheter opening.

Still referring to FIG. 5, in an unexpanded state, vascular implant may be ready for delivery into a patient for deployment at an aneurysm site. That is, an unexpanded state may refer to a state in which vascular implant 500 is configured to be delivered, which may be an as-cut or a crimped state. Base sections 509 and 511 and aneurysm section 517 of vascular implant 500 may be configured in a generally cylindrical form in the unexpanded state. More specifically, base rings 514, transition rings 510, 512, and aneurysm arcs 516 of vascular implant 500 may extend substantially around a circumference of vascular implant 500 in the unexpanded state such that vascular implant 500 includes a generally cylindrical form. As referred to here, substantially cylindrical means that although the aneurysm section 517 may be circumferentially discontinuous, due to aneurysm arcs 516 being separated by void 520, aneurysm section 517 nonetheless wraps substantially around the longitudinal axis 502. In an embodiment, one or more aneurysm arcs 516, or a geometric cord extending along an aneurysm arc 516 between aneurysm section holders 518, traverse an angle greater than about 300 degrees in the unexpanded state. For example, the traversed angle may be between about 320 to 360 degrees in the unexpanded state. More particularly, in an embodiment, a portion of aneurysm section 517 sweeps across an angle of about 330 degrees to 345 degrees between the aneurysm section holders 518 in both an expanded state and an unexpanded, laser-cut, state. In an unexpanded crimped state, the sweep angle may be close to 360 degrees, e.g., in a range of about 350 degrees to 360 degrees.

Geometrically, aneurysm section 517 may be described as having a contour of a longitudinal cylindrical segment, meaning that the profile wraps around a portion of a cylinder dissected by a longitudinal plane. In an embodiment, the longitudinal plane may be parallel to the longitudinal axis 502 such that a circumferential arc length at any point along aneurysm section 517 is approximately the same. In another embodiment, the longitudinal plane may be curvilinear such that the circumferential arc length varies along an axial length of aneurysm section 517.

Aneurysm section 517 may alternatively be considered to be the cylindrical shape surrounding a longitudinal slot formed in a cylindrical implant. For example, void 520 may be the longitudinal slot, and by forming void 520 with longitudinal aneurysm section holders 518, a cylindrical arc section may be formed, defining aneurysm section 517.

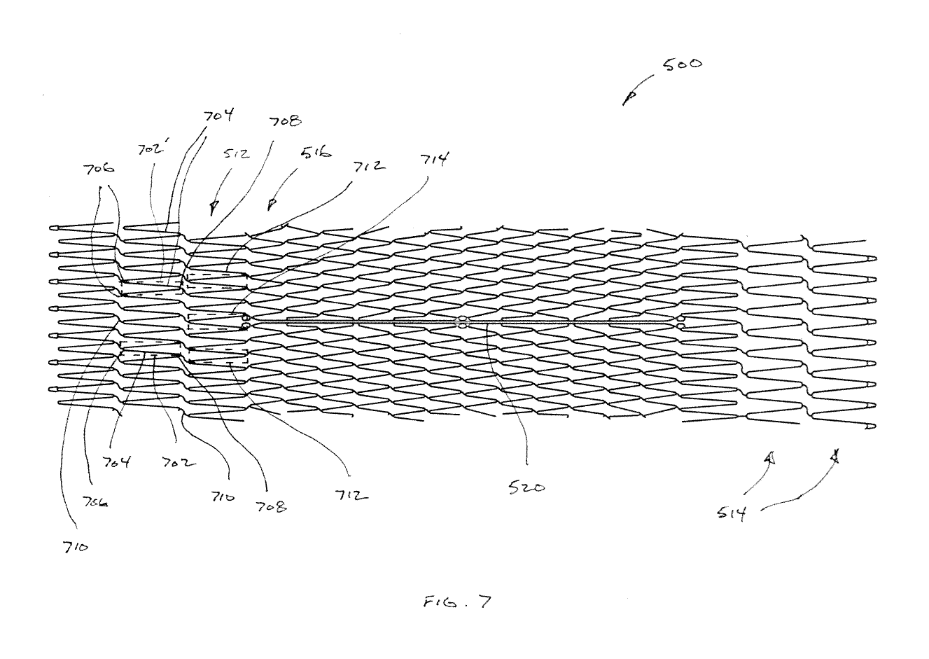

Referring to FIG. 6, a perspective view of a vascular implant in an expanded state is shown in accordance with an embodiment of the invention. In an embodiment, ring and arc scaffold structures of vascular implant 500 may be configured such that void 520 enlarges as vascular implant 500 expands. Base sections 509, 511, and aneurysm section 517 may expand toward generally cylindrical configurations in an expanded state. More particularly, a profile of vascular implant 500 may be generally cylindrical, just as a profile of a parent vessel extending across an aneurysm site is generally cylindrical, notwithstanding an aneurysm sac portion of the parent vessel. Accordingly, various rings of vascular implant 500, such as proximal transition ring 512, may expand to a larger ring diameter since ring undulations 522 maintain a contiguous and continuous structure around vascular implant 500 circumference. Similarly, various arcs of vascular implant 500, such as aneurysm arc 516, may expand to include a larger arc length between a first aneurysm section holder 602 and a second aneurysm section holder 604. In an embodiment, a geometric surface defined by void 520, e.g., a projected area between first aneurysm section holder 602 and second aneurysm section holder 604, may likewise increase. Thus, arc undulations 524 may maintain a contiguous, albeit circumferentially discontinuous structure around vascular implant 500. However, aneurysm section 517 may nonetheless include a substantially cylindrical profile in the expanded state. In an embodiment, one or more aneurysm arcs 516, or a geometric cord extending along an aneurysm arc 516 between aneurysm section holders 518, traverse an angle greater than about 300 degrees in the expanded state. For example, the traversed angle may be between about 320 to 360 degrees in the expanded state. More particularly, in an embodiment, a portion of aneurysm section 517 sweeps across an angle of about 330 degrees to 345 degrees between the aneurysm section holders 518 in the expanded state. Accordingly, although void 520 enlarges, aneurysm section 517 may nonetheless extend around substantially the same ratio of vascular implant 500 circumference in both the laser-cut, unexpanded, state and the expanded state.

Referring to FIG. 7, a flat pattern illustration of a vascular implant as described with respect to FIGS. 5-6 and having a void that enlarges during expansion is shown in accordance with an embodiment of the invention. More particularly, vascular implant 500 pattern is shown in a flattened configuration as if the contiguous lengths of rings and arcs are separated along a plane encompassing longitudinal axis 502 and a point diametrically opposed to aneurysm section holders 518. Vascular implant 500 may include a variety of undulations combined to allow void 520 to enlarge during expansion from the unexpanded state the expanded state.

In an embodiment, base rings, such as a base ring 514 in proximal base section 509, include one or more base undulation 702. Base undulation 702 may include adjacent base struts 704 having respective ends at proximal base joints 706 and a shared end at a distal base joint 708. Accordingly, base undulation 702 may form a generally v-shaped undulation pointing in a distal direction. Alternatively, base undulation 702′ may include adjacent base struts 704 having respective ends at distal base joints 708 and a shared end at a proximal base joint 706. Thus, base undulation 702′ may form a generally v-shaped undulation pointing in a proximal direction. Each base undulation 702 may be contiguous with an adjacent base undulation 702 to form base ring 514 extending continuously around vascular implant 500 circumference. Furthermore, base rings 514 of vascular implant 500 may be joined by one or more base connectors 710. Base connectors 710 may connect every base joint in adjacent base rings 514, thereby forming a closed-cell pattern, or at least one base joint in adjacent base rings 514 may not include a base connector 710, thereby forming an open-cell pattern. A distal-most base ring 514 in proximal base section 509, or a proximal-most base ring 514 in distal base section 511, may be joined with a respective transition ring by one or more base connector 710.

In an embodiment, transition rings, such as proximal transition ring 512, may include a variety of different undulation geometries. For example, proximal transition ring 512 may include one or more transition undulation 712 and one or more constraint undulation 714. Transition undulations 712 and constraint undulation 714 may have a same or different geometry. In an embodiment, transition undulations 712 connect with and determine in part a degree to which aneurysm arcs 516 expand as vascular implant 500 transitions from an unexpanded state to an expanded state. In an embodiment, constraint undulations 714 connect with and determine in part a degree to which aneurysm section holders 518 expand as vascular implant 500 transitions from an unexpanded state to an expanded state.

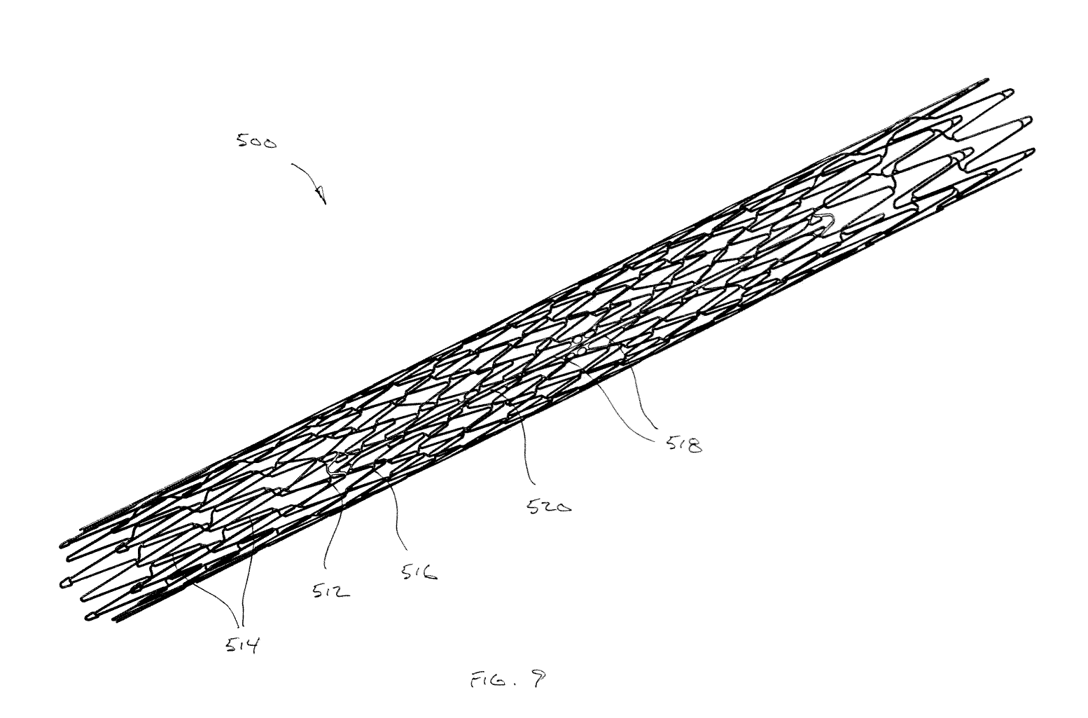

Referring to FIG. 8, a side view of a constraint undulation region of a vascular implant is shown in accordance with an embodiment of the invention. Constraint undulation 714 of a transition ring, such as proximal transition ring 512, may include constraint struts 802 extending between transition joints 804. For example, a first constraint strut 802 may extend proximally from one of a plurality of transition joints 804 near a proximal end of first aneurysm section holder 602, and a second constraint strut 802 may extend proximally from another, adjacent, transition joint 804 near a proximal end of second aneurysm section holder 604. Thus, in an embodiment, aneurysm section holders 518 extend from a constraint undulation 714 of each transition ring. More particularly, aneurysm section holders 518 may extend from respective transition joints 804 connected with constraint undulation 714. Constraint struts 802 extending proximally from the shared respective transition joints 804 may join each other at one of a plurality of transition joints 804 at a proximal end, thus forming a v-shaped constraint undulation 714.

In an embodiment, one or more transition undulation 712 of a transition ring, such as proximal transition ring 512, may be circumferentially adjacent to constraint undulation 714. For example, constraint undulation 714 may have a transition undulation 712 circumferentially lateral to each constraint strut 802. Similar to constraint undulation 714, each transition undulation 712 may include a plurality of struts, such as expansion struts 806. One expansion strut 806 may extend proximally from a transition joint 804 connected to first aneurysm section holder 602 and join an adjacent expansion strut 806 at one of a plurality of transition joints 804 at a proximal end.

Whereas transition undulations 712 and constraint undulations 714 of transition ring may be connected to an adjacent base ring 514 by one or more base connector 710, transition undulations 712 and constraint undulations 714 of transition ring may be connected to aneurysm section 517 by one or more transition connector 808. For example, a transition joint 804 joining a constraint strut 802 to an expansion strut 806 may be connected to one of a plurality of arc joints 810 that similarly connect an aneurysm section holder 602, 604, to an arc strut 812.

In an embodiment, every transition joint 804 of a transition ring is connected to a respective adjacent arc joint 810 by a transition connector 808, thereby creating a closed-cell pattern between the transition ring and aneurysm section 517. Thus, vascular implant 500 may include a same number of distal transition joints 804 in distal transition ring 510 as there are proximal arc joints 810 in aneurysm arc 516. For example, distal transition ring 510 may have fourteen distal transition joints 804 connected to fourteen arc joints 810 by fourteen transition connectors 808. Alternatively, vascular implant 500 may include different numbers of transition joints 804 and adjacent arc joints 810 and/or may not include transition connectors 808 connecting every transition joint 804 with an arc joint 810, thereby creating an opened-cell pattern.

In an embodiment, each undulation, e.g., base undulation 702, transition undulation 712, constraint undulation 714, or arc undulation 524, includes a respective composite stiffness. A composite stiffness of a respective undulation may correlate with a degree to which a heat set scaffold structure reduces in diameter when an external crimping load is applied. For example, vascular implant 500 may be formed from a tube, e.g., by laser cutting a tube of superelastic nickel titanium alloy, and then heat setting the laser cut structure in the expanded state. However, prior to delivering vascular implant 500, an inward radial load may be applied by a crimping device to reduce the vascular implant 500 diameter for loading into a delivery catheter. Composite stiffness of an undulation may determine whether undulation joints are deflected during crimping. Likewise, composite stiffness of an undulation may determine whether undulation joints are able to flex outward, e.g., in a case where a secondary device is tracked through a cell including the undulation in a bifurcated application.

A composite stiffness of an undulation may depend on numerous factors. For example, the composite stiffness may depend on an axial length between a proximal and distal joint of the undulation, e.g., between a proximal transition joint 804 and a distal transition joint 804. Composite stiffness may further depend on a width or thickness at one or more locations along struts of the undulation, e.g., a strut width of constraint strut 802 or expansion strut 806. Similarly, composite stiffness may depend on a width or thickness at an undulation joint, e.g., a width of a joint joining constraint struts 802 of constraint undulation 714 or of transition joint 804 joining expansion struts 806 of transition undulation 712. In an embodiment, composite stiffness depends on various radii of the undulation, e.g., an inner radius of transition joints 804 of the undulation. Composite stiffness may also depend on material properties of vascular implant 500, such as a material used to form undulations of vascular implant 500, e.g., stainless steel or superelastic nickel titanium, or a heat treatment used during the manufacture of vascular implant 500. Accordingly, composite stiffness of an undulation may be modified by controlling numerous properties of vascular implant 500.

In an embodiment, vascular implant 500 having fourteen crowns at either end of an aneurysm arc 516 includes void 520 formed between two aneurysm section holders 602, 604. Void 520 may span about 26 degrees. More particularly, the void 520 formed by two aneurysm section holders 602, 604, may be in a range of between about 15 degrees to 30 degrees so as to control the opening of aneurysm section 517. Both the dimension and design attributes of vascular implant 500 may be tailored such that heat setting vascular implant 500 to achieve the target opening 520 becomes easier. For example, a heat treat mandrel used during manufacturing of vascular implant 500 may be designed to facilitate achieving the target opening of void 520.

In an embodiment, a composite stiffness of constraint undulation 714 may be controlled to be equal to a composite stiffness of transition undulations 712 in a transition ring. For example, the axial length, strut widths, strut thicknesses, and geometry of transition undulation 712 and constraint undulation 714, as represented in FIG. 8, may be the same such that composite stiffness of transition undulation 712 and constraint undulation 714 are equal. Accordingly, transition undulation 712 and constraint undulation 714 may reduce in size under a crimping load, and expand in size upon deployment from a delivery catheter 406, by substantially a same amount. For example, distal transition joints 804 of transition undulation 712 and constraint undulation 714 in proximal transition ring 512 may approach each other by a same amount, and separate by a same distance or angle, as vascular implant 500 transitions from an expanded state, i.e., after heat setting, to an unexpanded state, i.e., after crimping, and then back to an expanded state, i.e., after deployment. Furthermore, given that constraint undulations 714 and transition undulations 712 have essentially the same composite stiffness, as the undulations are crimped or expanded, aneurysm section holders 518 connected to constraint undulation 714 may expand or crimp to a same degree as arc undulations 524 connected to transition undulations 712, thereby allowing void 520 to enlarge when vascular implant 500 transitions from the unexpanded state to the expanded state.

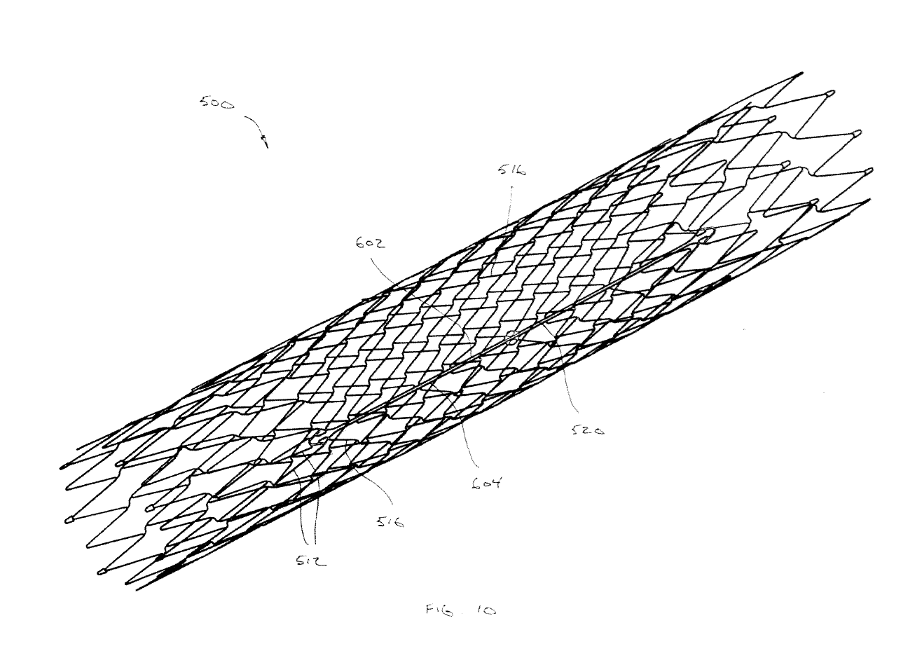

Referring to FIG. 9, a perspective view of a vascular implant in an unexpanded state is shown in accordance with an embodiment of the invention. In an embodiment, vascular implant 500 includes similar geometry and elements as compared to the embodiment shown in FIG. 5, above. More specifically, vascular implant 500 may include a base section having a plurality of base rings 514 that are connected to an aneurysm section having a plurality of aneurysm arcs 516 through one or more transition ring, e.g., proximal transition ring 512. Furthermore, while transition rings may be circumferentially continuous, aneurysm arc 516 may connect at opposite ends to respective aneurysm section holders 518, separated by void 520. Thus, aneurysm arc 516 may be circumferentially discontinuous in the unexpanded state. Although discontinuous, aneurysm arc 516 may extend substantially around the circumference of vascular implant 500. However, in contrast to the embodiment shown in FIG. 5, in an embodiment, ring and arc scaffold structures of vascular implant 500 may be configured such that void 520 remains substantially the same as vascular implant 500 expands.

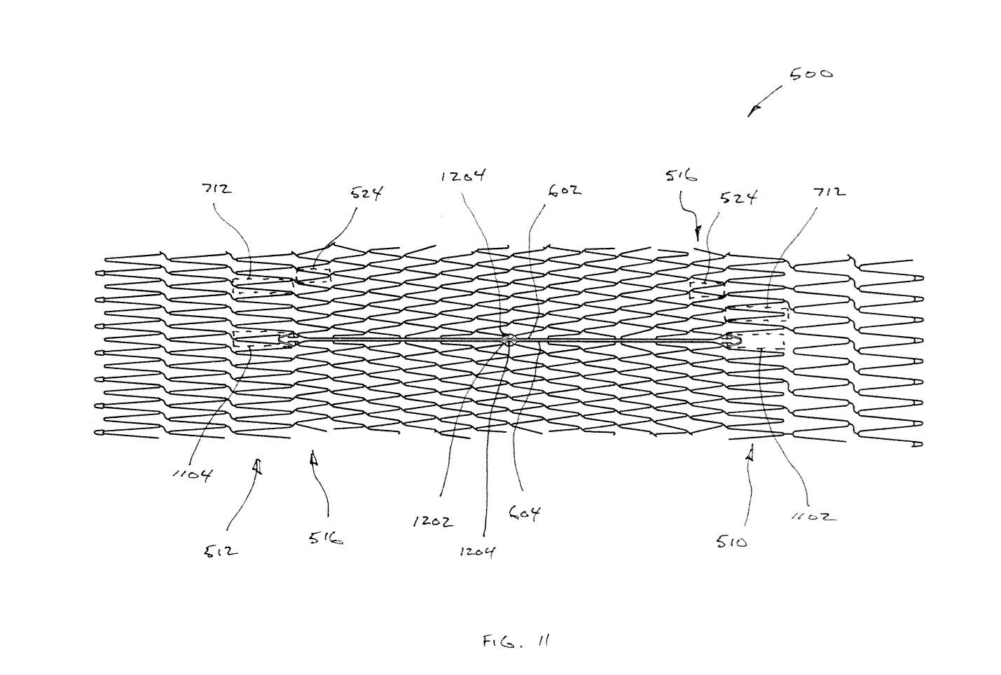

Referring to FIG. 10, a perspective view of a vascular implant in an expanded state is shown in accordance with an embodiment of the invention. Similar to the embodiment shown in FIG. 6, base sections and aneurysm section may expand toward generally cylindrical configurations as vascular implant 500 transitions to the expanded state. More particularly, ring scaffold structures of vascular implant 500, such as proximal transition ring 512, may expand to a larger ring diameter while ring undulations 522 maintain a contiguous and continuous structure around vascular implant 500 circumference. Similarly, arc scaffold structures of vascular implant 500, such as aneurysm arc 516, may expand to include a larger arc length between first aneurysm section holder 602 and second aneurysm section holder 604. However, in an embodiment, a geometric surface defined by void 520, e.g., a projected area between first aneurysm section holder 602 and second aneurysm section holder 604, may remain the same through expansion of vascular implant 500. Thus, arc undulations 524 may maintain a circumferentially discontinuous structure around vascular implant 500 and include a substantially cylindrical profile in the expanded state. In an embodiment, one or more aneurysm arcs 516, or a geometric cord extending along an aneurysm arc 516 between aneurysm section holders 602, 604, traverse an angle greater than about 300 degrees in the expanded state. For example, the traversed angle may be between about 330 to 360 degrees in the expanded state. More particularly, in an embodiment, a portion of aneurysm section 517 sweeps across an angle of about 355 degrees between the aneurysm section holders 602, 604, in the expanded state. Furthermore, the traversed angle may be the same in both the unexpanded state and the expanded state. For example, the aneurysm section 517 may sweep across an angle of about 356 degrees between the aneurysm section holders 518 before and after vascular implant 500 is expanded within a target vessel.

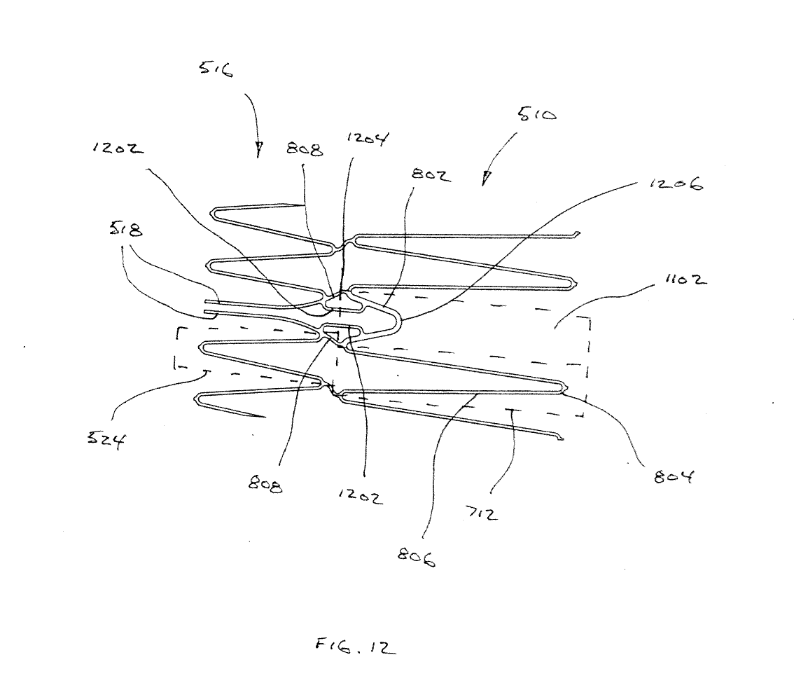

Referring to FIG. 11, a flat pattern illustration of a vascular implant as described with respect to FIGS. 9-10 is shown in accordance with an embodiment of the invention. In an embodiment, vascular implant 500 includes constraint undulations connected with proximal and distal ends of first aneurysm section holder 602 and second aneurysm section holder 604. Furthermore, constraint undulations may have different geometries. For example, constraint undulations of vascular implant 500 may include distal constraint undulation 1102 and proximal constraint undulation 1104, each of which includes different geometry. In an embodiment, distal constraint undulation 1102 and proximal constraint undulation 1104 include composite stiffness greater than transition undulations 712 in respective transition rings such that aneurysm section holders 602, 604, connected to distal constraint undulation 1102 and proximal constraint undulation 1104 remain circumferentially stationary during the heat treat process and thus during both crimping and expansion of vascular implant 500. More particularly, crimping vascular implant 500 does not substantially reduce void 520 between aneurysm section holders 602, 604, and expanding vascular implant 500 does not substantially increase void 520 between aneurysm section holders 602, 604. Although “distal” and “proximal” qualifiers are applied to constraint undulation embodiments herein, the constraint undulations may be located and combined in any manner as shall be apparent to one skilled in the art.

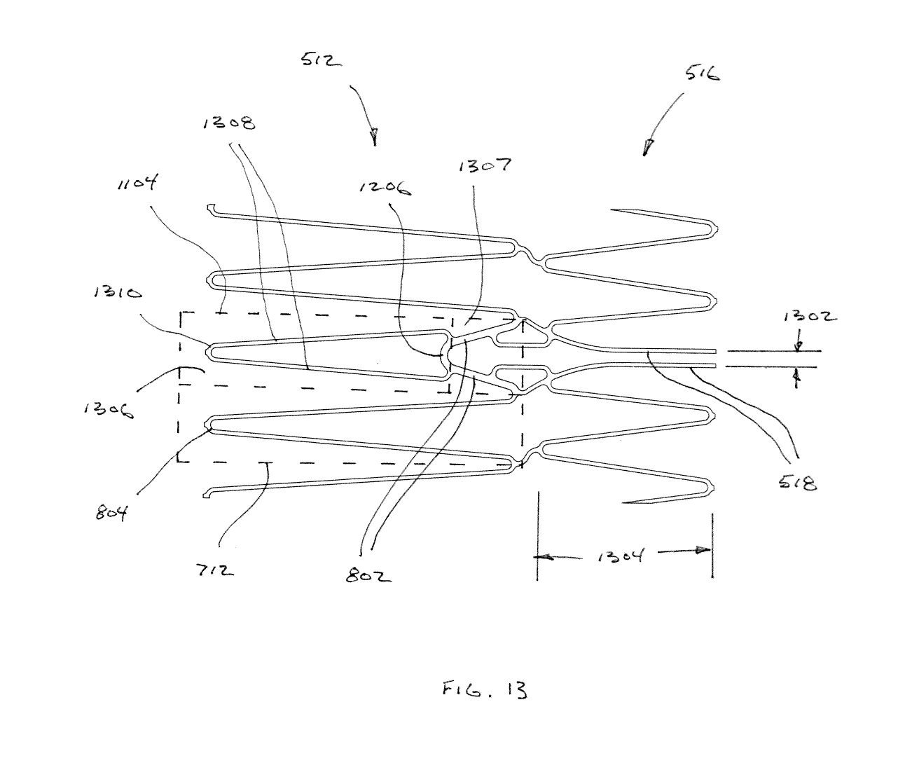

Referring to FIG. 12, a side view of a distal constraint undulation region of a vascular implant is shown in accordance with an embodiment of the invention. In an embodiment, distal constraint undulation 1102 includes a composite stiffness greater than transition undulations 712 in a same transition ring such that aneurysm section holders 518 expand or contract to a lesser degree than arc undulations 524 of aneurysm arc 516. For example, constraint undulation 1102 may include a higher composite stiffness than a composite stiffness of an adjacent transition undulation 712. Furthermore, a composite stiffness of constraint undulation 1102 may be higher than a respective composite stiffness of each transition undulation 712 in the shared transition ring. Accordingly, constraint struts 802 of constraint undulation 1102 may be at least one of shorter or wider than expansion struts 806 of transition undulation 712, such that constraint undulation 1102 resists expansion from radial loading more than transition undulation 712.

Composite stiffness of constraint undulations may be varied in numerous manners. For example, constraint struts 802 may be shorter, e.g., may have lengths in an axial direction in a range of about 0.0178-inch to 0.0266-inch, while expansion struts 806 may have lengths in the axial direction in a range of about 0.0627-inch to 0.0941-inch. Similarly, constraint struts 802 may be wider, e.g., may be in a range of about 0.0016-inch to 0.0020-inch, than expansion struts 806, which may be in a range of about 0.0008-inch to 0.0012-inch. More particularly, expansion struts 806 may have a width of about 0.0010-inch. Further still, a width of constraint joint 1206 joining constraint struts 802 of constraint undulation 1102 may be wider, e.g., may be in a range of about 0.0016-inch to 0.0020-inch, than transition joints 804 joining expansion struts 806 of transition undulations 712, which may be in a range of about 0.0008-inch to 0.0012-inch. More particularly, transition joints 804 may have a width of about 0.0010-inch.

Referring to FIG. 13, a side view of a proximal constraint undulation region of a vascular implant is shown in accordance with an embodiment of the invention. Proximal constraint undulation 1104 may include geometry similar to distal constraint undulation 1102. For example, proximal constraint undulation 1104 may also include a composite stiffness greater than adjacent transition undulations 712, such that radial expansion of vascular implant 500 results in expansion of aneurysm arc 516, with minimal corresponding expansion of void width 1302 between aneurysm section holders 518. More specifically, void width 1302, which may be measured along any distance of aneurysm section holders 518, including arc segment longitudinal length 1304, may remain approximately the same as vascular implant 500 transitions from the unexpanded state to the expanded state.

In an embodiment, void width 1302 may be minimized given the available manufacturing processes. For example, in the case of a laser cut implant, void width 1302 may have a dimension on the order of a laser kerf width. Thus, void width 1302 may be in a range of about 0.0005-inch to 0.005-inch. More particularly, void width 1302 may be approximately in the range of about 0.00075-inch to 0.0026-inch. For example, void width 1302 may be about 0.0012-inch. This width may also vary depending upon material removal after subjecting the implant to electropolishing during manufacturing. Alternatively, void width 1302 may be sized as a ratio of implant circumference. For example, void width 1302 may be less than about ten percent of the circumference of vascular implant 500 in the unexpanded state and the expanded state. More particularly, void width 1302 may be in a range of about one percent to seven percent of the circumference of vascular implant 500 in the unexpanded state and the expanded state. Since void 520 may be formed with a signal pass of a laser cut, void 520 may have a straight or curved path with a same void width 1302 over the entire path.

In an embodiment, proximal transition ring 512 may also include a stack undulation 1306 connected with constraint undulation 1307. Thus, constraint undulation 1104 may be considered to include the combination of stack undulation 1306 and constraint undulation 1307. Stack undulation 1306 may include a plurality of stack struts 1308 extending proximally from constraint undulation 1307 to a stack joint 1310. More particularly, stack struts 1308 may have distal ends connected to constraint undulation 1307 adjacent to constraint joint 1206 of constraint undulation 1307. For example, the distal ends of stack struts 1308 may connect at a location along respective constraint struts 802. Furthermore, stack struts 1308 may have a length such that stack joint 1310, where stack struts 1308 join, is circumferentially aligned with proximal transition joints 804 of one or more transition undulations 712 in proximal transition ring 512. Accordingly, an overall length of the combination of constraint undulation 1307 and stack undulation 1306 may be approximately equal to an overall length of transition undulations 712 in proximal transition ring 512.

In an embodiment, stack undulation 1306 allows for constraint undulation 1307 to be connected with a base ring 514 adjacent to proximal transition ring 512. For example, without stack undulation 1306, a gap may exist between constraint undulation 1307 and an adjacent base ring 514. In other words, implant 500 may have an open-cell pattern in the constraint undulation region. This possibility is illustrated in FIG. 12, above. In the embodiment of FIG. 12, retrieval of vascular implant 500 after deployment from a delivery catheter is not challenging. For example, constraint undulation 1102 may flare outward as it is deployed from the catheter. However, it may not become caught on a delivery catheter during retrieval since the direction of retrieval is toward proximal side and the open cell exists on distal side. By contrast, retrieval of an open-cell pattern near a proximal constraint undulation region may be challenging. Thus, as illustrated in FIG. 13, with constraint undulation 1307 connected to the adjacent base ring 514 by stack undulation 1306 in a closed-cell pattern, a smooth transition is provided between a transition ring and an adjacent base ring 514, thus allowing for retrieval without snagging the base ring 514 on the delivery catheter 406.

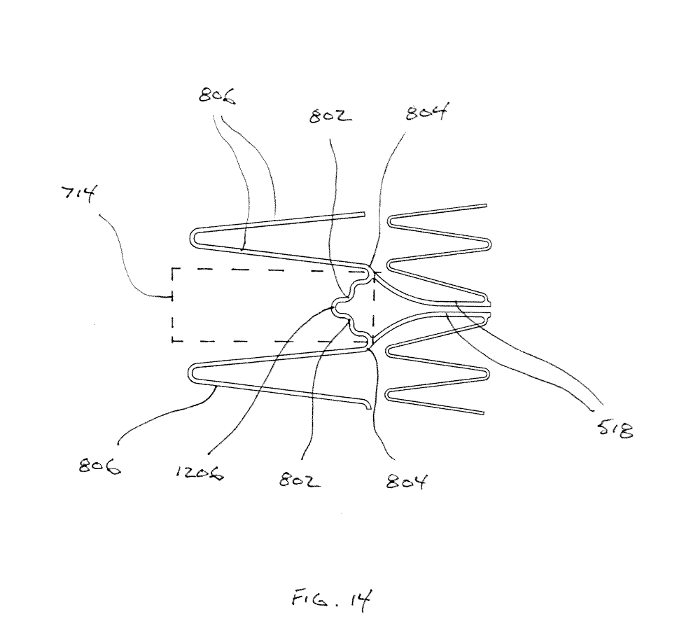

Referring to FIG. 14, constraint undulations 714 may be located and combined to impart the desired expansion characteristics to vascular implant 500. For example, a constraint undulation 714 with lesser composite stiffness may be incorporated in vascular implant 500 at a proximal or a distal end of aneurysm section holders 518, and a constraint undulation 714 with higher composite stiffness may be incorporated at an opposite end of aneurysm section holders 518. Such an embodiment may provide, for example, a void 520 that remains substantially closed near an end of aneurysm section holders 518, but opens near another end of aneurysm section holders 518. Thus, void 520 may be more easily crossed near the other end of vascular implant 500, such as by a guidewire passing through vascular implant 500 to access a side branch vessel.

Still referring to FIG. 14, a side view of a constraint undulation region of a vascular implant is shown in accordance with an embodiment of the invention. In addition to being combined differently to tune the expansion of void 520, constraint undulation 714 geometry may also be varied to provide for varying degrees of expansion. In an embodiment, constraint undulation 714 includes a plurality of constraint struts 802 that undulate between respective transition joints 804. For example, constraint undulation 714 may include two constraint struts 802, each of which undulates from a transition joint 804 joining the constraint strut 802 with an adjacent expansion strut 806. The two constraint struts 802 may extend to meet at a constraint joint 1206. In an embodiment, each constraint strut 802 undulates through a single wave that extends along a concave inward path, the paths being symmetric such that constraint undulation 714 in combination with aneurysm section holders 518 forms a generally clover-shaped profile.

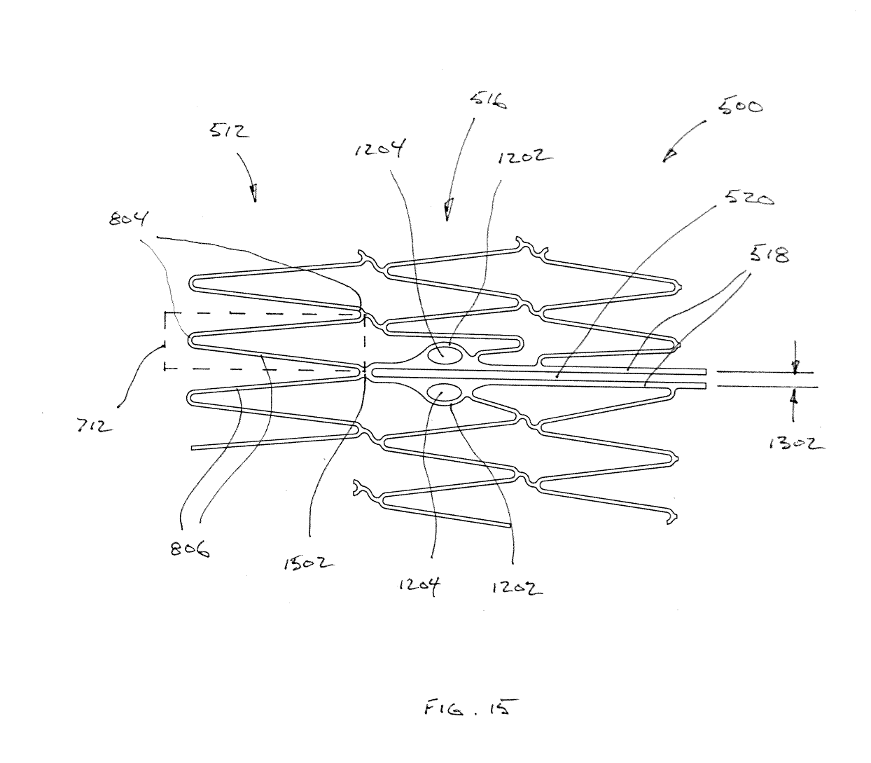

Referring to FIG. 15, a side view of a constraint terminal region of a vascular implant is shown in accordance with an embodiment of the invention. In an embodiment, vascular implant 500 includes a constraint terminal 1502 joining expansion struts 806 to aneurysm section holders 518. Constraint terminal 1502 may be a joint that provides sufficient stiffness to replace constraint undulation 714. For example, constraint terminal 1502 may have a width that is greater than a width of transition joints 804 in adjacent transition undulations 712. In an embodiment, the width of constraint terminal 1502 is in an axial direction, and is greater than about twice a width of transition joints 804. For example, whereas transition joints 804 may have a strut width of approximately 0.0010-inch, constraint terminal 1502 may have a width in an axial direction of approximately 0.0018-inch. Similarly, constraint terminal 1502 may have a width in a circumferential direction of at least the same distance as the axial width. For example, constraint terminal 1502 may have a circumferential width of approximately 0.0018-inch or more. In an embodiment, constraint terminal 1502 includes a circumferential width of about 0.003-inch. Accordingly, constraint terminal 1502 may be comparatively stiff in relation to transition joints 804 in the same transition ring, and thus, expansion of expansion struts 806 connecting with constraint terminal 1502 may be comparatively less than expansion of other expansion struts 806 in the transition ring. Similarly, the constraint terminal 1502 may terminate two aneurysm section holders 518 into single joint with a stiffness sufficient to limit expansion of the aneurysm section holders 518, such that void width 1302 remains substantially the same when vascular implant 500 transitions from a heat treat process to the crimped state to the expanded state.

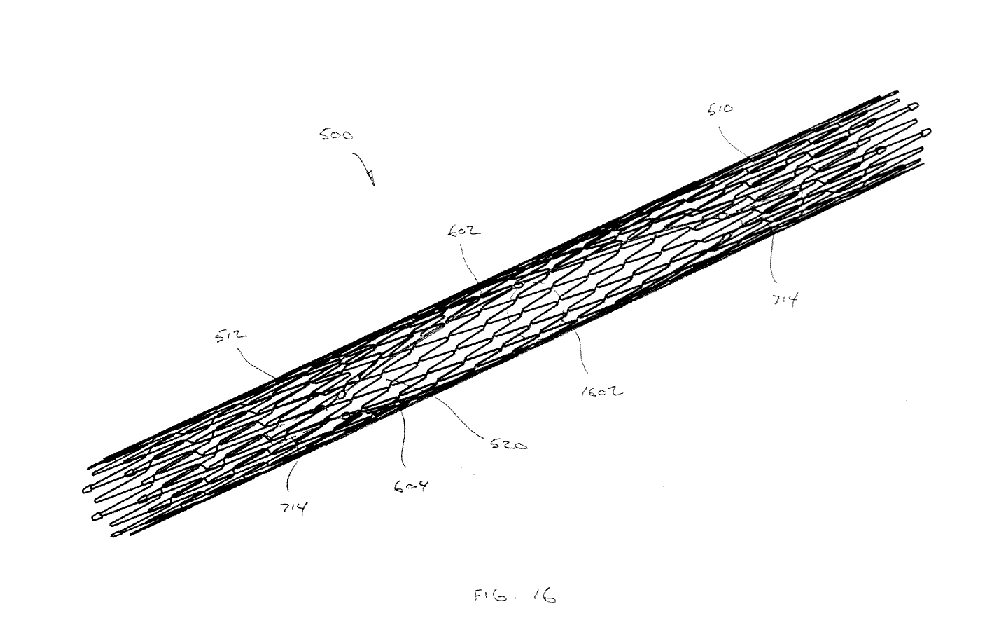

Referring to FIG. 16, a perspective view of a vascular implant in an unexpanded state is shown in accordance with an embodiment of the invention. In an embodiment, vascular implant 500 may include first aneurysm section holder 602 and second aneurysm section holder 604 extending between unexpanded distal transition ring 510 and proximal transition ring 512, such that void 520 includes a port 1602. For example, each aneurysm section holder may include an arcuate shape such that a circular port 1602 may be fit therebetween. More particularly, first aneurysm section holder 602 may have a generally bow-shaped path between respective constraint undulations 714 of distal transition ring 510 and proximal transition ring 512. Second aneurysm section holder 604 may mirror the path of first aneurysm holder, and therefore, void 520 may have a generally eye-shaped, or elliptical profile. Accordingly, circular port 1602 may represent only a portion of void 520 defined between aneurysm section holders.

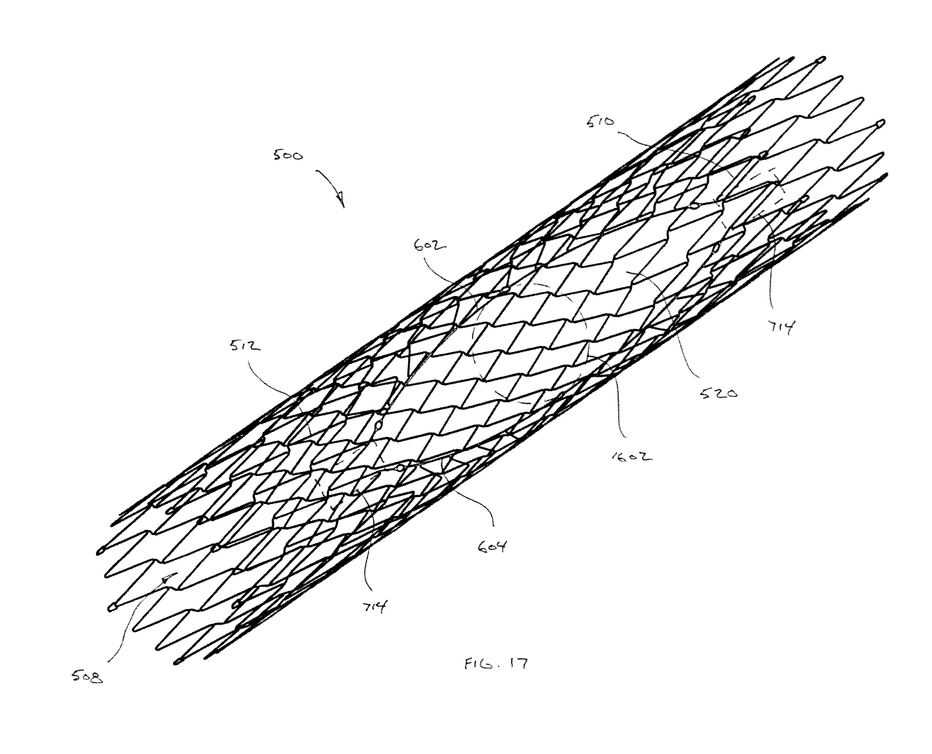

Referring to FIG. 17, a perspective view of a vascular implant in an expanded state is shown in accordance with an embodiment of the invention. In an embodiment, distal transition ring 510 and proximal transition ring 512 increase in diameter as vascular implant 500 transitions from the unexpanded state to the expanded state. Furthermore, constraint undulations 714 may be configured to expand to some degree, resulting in separation of limit expansion of aneurysm section holders 602, 604. Accordingly, void 520, and therefore port 1602, may increase in size and shape between the unexpanded state and the expanded state. Alternatively, constraint undulations 714 may be configured to limit expansion of aneurysm section holders 602, 604. Accordingly, void 520, and therefore port 1602, may remain approximately the same size and shape between the unexpanded state and the expanded state.

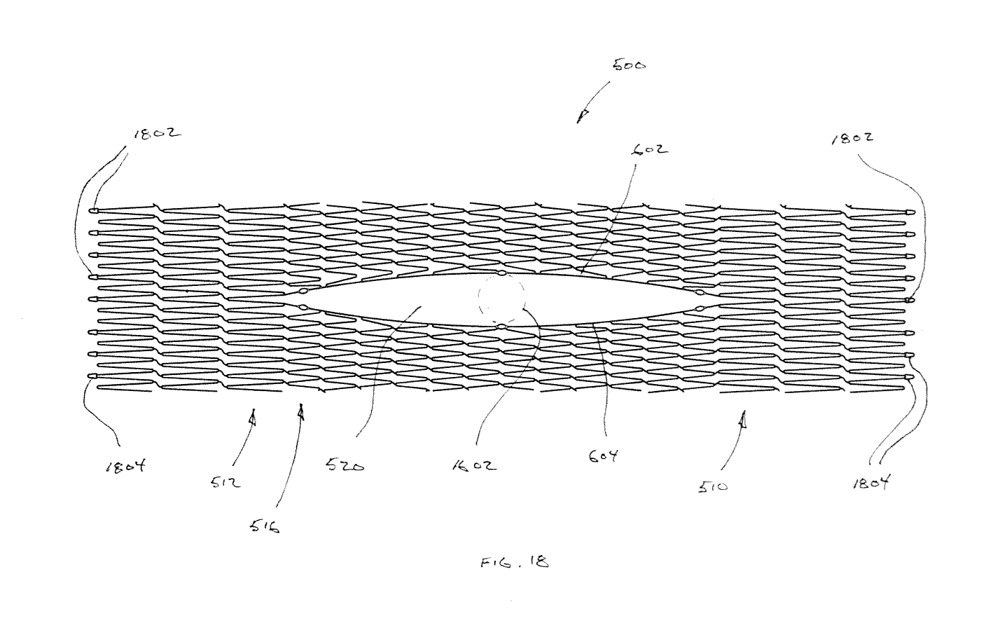

Referring to FIG. 18, a flat pattern view of a vascular implant as described with respect to FIGS. 16-17 is shown in accordance with an embodiment of the invention. In an embodiment, port 1602 may provide an opening between an inner lumen of vascular implant 500 and a surrounding environment. For example, port 1602 and/or void 520 may have a projected area sufficient to allow a guidewire or catheter device to be tracked from a main branch in which vascular implant 500 is positioned into a side branch toward which void 520 is aligned. Typical guidewire and catheter devices used during minimally invasive catheterization procedures, such as balloon angioplasty and self-expanding stent deployment procedures, may range up to about 2 mm in diameter. Thus, void 520 and/or port 1602 may include projected areas with a crossing dimension of between about 1-2 mm. In an embodiment, port 1602 is sized smaller than an anticipated secondary device, such as in a range of between about 0.2-1 mm, but aneurysm section holders 602, 604 are sufficiently flexible to allow the secondary device to widen void 520 as it is advanced through port 1602 over a guidewire. Accordingly, port 1602 may be sized to be larger than a profile diameter of an anticipated guidewire used for crossing into a side branch vessel, but smaller than a profile diameter of the anticipated secondary device. More particularly, void 520 and/or port 1602 may be sized and configured to complement a range of secondary medical devices.

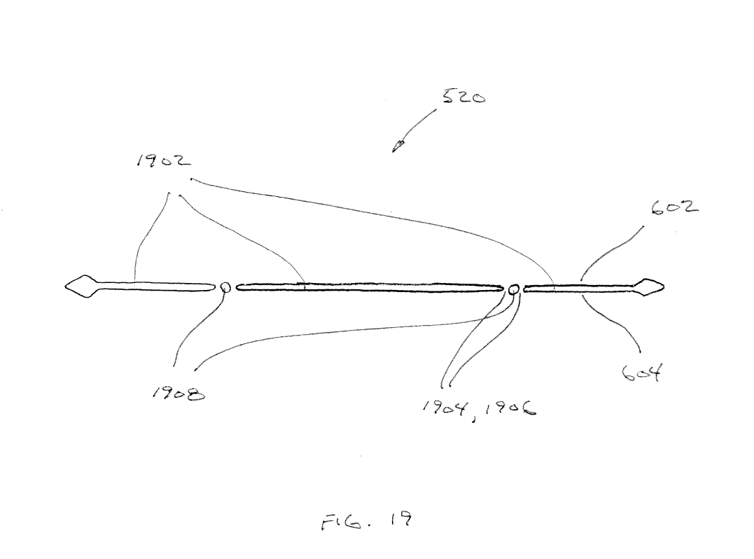

Referring to FIG. 19, a pictorial view of a void configuration is shown in accordance with an embodiment of the invention. In an embodiment, void 520 is partitioned into a plurality of void segments 1902 between constraint undulations at proximal and distal ends of aneurysm section holders. For example, one or more radial connector 1904 may connect first aneurysm section holder 602 to adjacent second aneurysm section holder 604. Therefore, void 520 between aneurysm section holders may be maintained by radial connectors 1904, in addition to the constraining forces of constraint undulations. However, although radial connectors 1904 may maintain a close separation between aneurysm section holders, void 520 may still allow aneurysm section holders to move relative to each other, e.g., by being splayed outward by a crossing secondary device or by wrapping over each other to prevent oval deformation in a tortuous anatomy.

In an embodiment, one or more intermediate aneurysm marker holder is formed in one or more radial connector 1904. For example, intermediate void marker holders 1906 may be spaced along void 520 at known distances, e.g., at distances of one-third of an axial length of void 520. Each intermediate void marker holder 1906 may be loaded with a void marker 1908. Thus, intermediate void marker holder 1906 locations may be viewed under fluoroscopy or other imaging modalities to ascertain an orientation of void 520 relative to an anatomy, or to allow for a rough measurement of the anatomy to be made by comparing the anatomy to the distances between void markers 1908. Void markers 1908 may include radiopaque material that may be stamped, injected, crimped, sputtered, or otherwise loaded into or onto intermediate void marker holders 1906. Markers for indicating an in vivo orientation of vascular implant 500 may facilitate accurate positioning and deployment relative to an aneurysm, and thus, additional marker configurations are described further below.

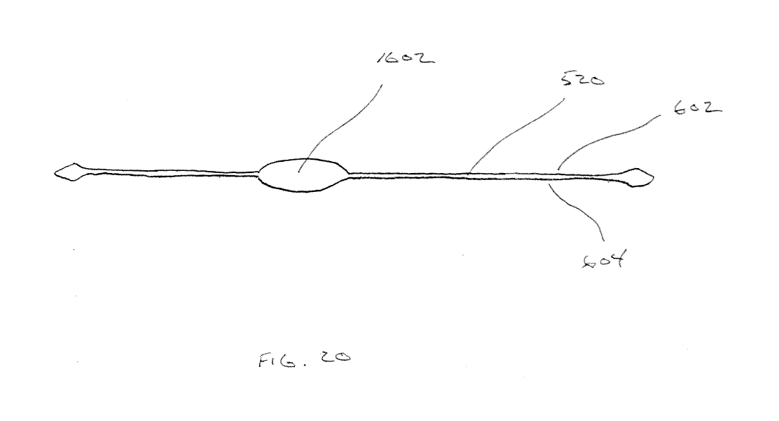

Referring to FIG. 20, a pictorial view of a void configuration is shown in accordance with an embodiment of the invention. In an embodiment, port 1602 may be closely defined by aneurysm section holders 602, 604, surrounding void 520. More particularly, whereas port 1602 was described above as effectively including an imaginary perimeter drawn within void 520, port 1602 may instead be defined by a portion of aneurysm section holder length. For example, aneurysm section holders may curve abruptly to form an elliptical, circular, or other curvilinear shape at a location between proximal and distal constraint undulations. Accordingly, port 1602 of a specific size and shape may be defined within the shape formed by aneurysm section holders. In an embodiment, port 1602 may be elliptical and defined between aneurysm section holders at a point approximately half, or alternatively one-third, of the distance between proximal and distal constraint undulations.

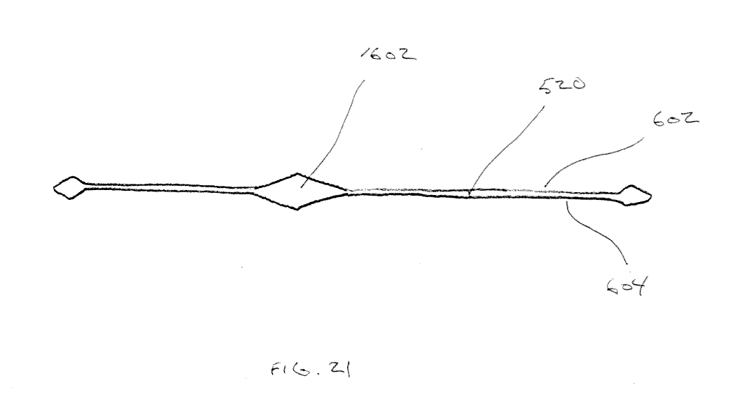

Referring to FIG. 21, a pictorial view of a void configuration is shown in accordance with an embodiment of the invention. In an embodiment, aneurysm section holders 602, 604, may follow a path that forms a polygonal void 1602. For example, aneurysm section holders may angulate away from a longitudinal axis along void 520 to create a diamond-shaped port 1602 at a point approximately half, or alternatively one-third, of the distance between proximal and distal constraint undulations. Aneurysm section holders may form other port 1602 shapes, including rectilinear shapes such as squares or rectangles, or other polygonal shapes, including triangles, hexagons, etc.

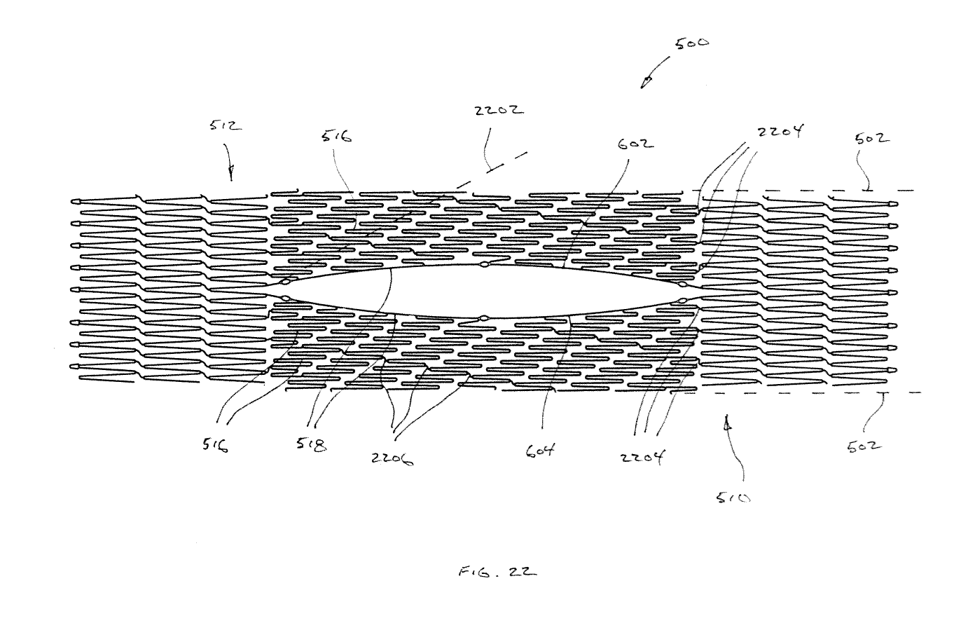

Referring to FIG. 22, a flat pattern illustration of a slanted aneurysm section pattern of a vascular implant is shown in accordance with an embodiment of the invention. In an embodiment, aneurysm section includes a plurality of aneurysm arcs 516 that, rather than extending between first aneurysm section holder 602 and second aneurysm section holder 604 along a straight circumferential direction, extend between aneurysm section holders along a helical path. That is, an arc axis 2202 through a flattened aneurysm arc 516 may extend in a slanted direction relative to longitudinal axis 502. As such, at least one aneurysm arc 516 of aneurysm section may extend between an aneurysm section holder and a transition ring, such as distal transition ring 510. Thus, a first end of aneurysm arc 516 may connect with aneurysm section holder 602 and a second end of aneurysm arc 516 may connect with distal transition ring 510 at aneurysm arc connector 2204. In an embodiment, although aneurysm arcs 516 may be arranged in a slanted pattern relative to longitudinal axis 502, a plurality of arc connectors 2206 that interconnect adjacent aneurysm arcs 516 of aneurysm section 517 may be generally aligned in an axial direction in a more or less staggered matter.

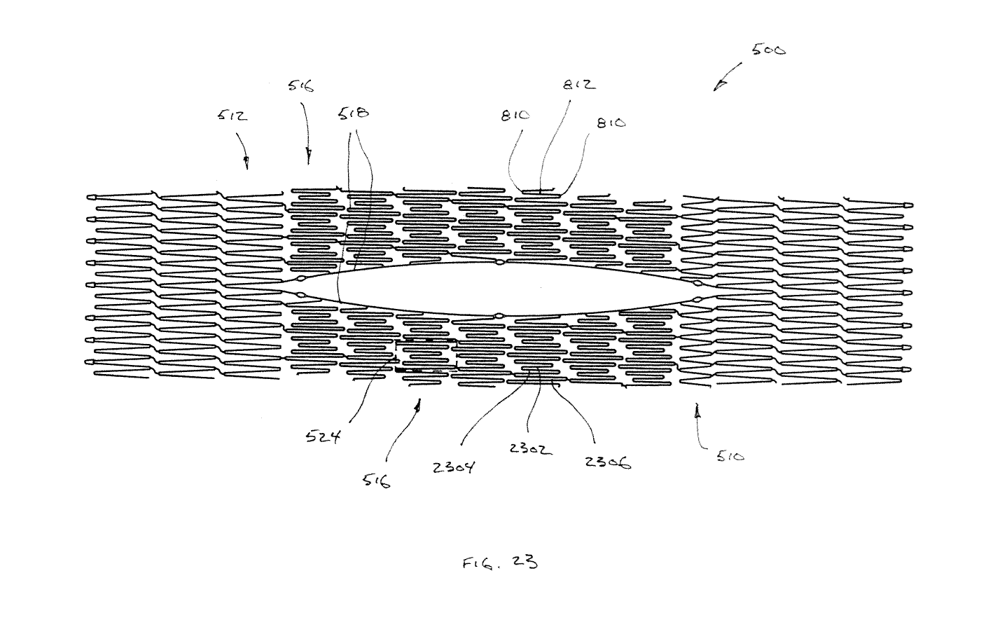

Referring to FIG. 23, a flat pattern illustration of an hourglass aneurysm section pattern of a vascular implant is shown in accordance with an embodiment of the invention. In an embodiment, undulations within respective rings or arcs of vascular implant 500 may vary circumferentially. For example, arc undulations 524 of aneurysm arcs 516 within aneurysm section may have varying axial lengths around a circumference of vascular implant 500. For example, aneurysm arc 516 may include arc undulations 524 that variously include short arc strut 2302, medium arc strut 2304, and long arc strut 2306. More particularly, in an embodiment, each arc undulation 524 includes arc struts 812 attached at arc joints 810 and reducing in length from a longest length to a shortest length and then increasing from the shortest length to the longest length. Thus, each arc undulation 524 may have a generally hour glass-shaped configuration. Arc undulation 524 may include a number of such undulations connected contiguously between aneurysm section holders 518, such that aneurysm section includes a circumferentially alternating pattern.

In an embodiment, aneurysm section may include a symmetric pattern about a central plane. For example, as described above relative to vascular implant 500 flat patterns, a central plane may be defined as encompassing longitudinal axis 502 and a point circumferentially opposite of void 520 between aneurysm section holders 518. Thus, each aneurysm arc 516 circumferentially disposed between aneurysm section holders 518 may be divided into a first arc pattern, e.g., between first aneurysm section holder 602 and the central plane, and a second arc pattern, e.g., between second aneurysm section holder 604 and the central plane. In an embodiment, the first arc pattern may be symmetric with the second arc pattern. For example, first arc pattern may be a minor image of second arc pattern. This feature may be useful and practical, e.g., when a symmetric heat treat pattern is desired.

Alternatively, the first arc pattern may be symmetric with the second arc pattern over only a portion of their lengths. More particularly, first arc pattern may have an arc length beginning at first aneurysm section holder 602 and second aneurysm section holder 604 may have the same arc length beginning at second aneurysm section holder 604. Furthermore, first arc pattern may be a minor image of second arc pattern over a portion of the arc length, e.g., over at least about three-quarters of the arc length. However, it may be that if first arc pattern and second arc pattern are completely symmetric, the arc patterns would not converge at the central plane, and thus, aneurysm arc 516 would be non-contiguous. Accordingly, one or both of first arc pattern and second arc pattern may be modified to ensure that the arc patterns meet at the central plane and aneurysm arc 516 is contiguous. For example, an angle between arc struts 812 may be widened or narrowed over a few arc undulations 524 near the central plane to allow for a smooth pattern transition to be made between first arc pattern and second arc pattern.

Referring again to FIG. 15, in an embodiment, vascular implant 500 includes one or more aneurysm marker holders 1202 to hold respective aneurysm markers 1204. Aneurysm markers 1204 may be provided at one or more location along aneurysm section holders 518 to indicate a location of void 520 relative to anatomy of a patient. For example, referring again to FIG. 12, aneurysm marker holders 1202 may be located circumferentially between transition connectors 808 connecting constraint undulation 1102 with aneurysm section holders 518. Accordingly, aneurysm marker holders 1202 may be laterally between constraint struts 802. Alternatively, aneurysm marker holders 1202 may be laterally between aneurysm section holders 518 near constraint strut 802. Thus, aneurysm marker holders 1202 may be integrally formed with other features of vascular implant 500 to fit within gaps of implant undulations such that aneurysm marker holders 1202 do not prevent or substantially disrupt crimping of the implant undulations to a delivery diameter in the unexpanded state.

Various other embodiments and configurations for aneurysm markers 1204 are also contemplated. For example, referring to FIG. 11, aneurysm marker holders 1202 may be located at one or more positions along aneurysm section holders 602, 604, such as near the middle of aneurysm section holders. Furthermore, aneurysm marker holders 1202 may be circumferentially opposite of void 520 from aneurysm section holders 518. Thus, aneurysm markers 1204 in aneurysm marker holders 1202 may be viewed to identify both rotational orientation of void 520 relative to an anatomy of interest, as well as axial orientation of void 520 relative to the anatomy. That is, by aligning aneurysm markers 1204 near the middle of aneurysm section holders 518 with an aneurysm, it may be inferred that aneurysm section is axially aligned with the aneurysm.

Similarly, aneurysm marker holders 1202 may be located near the ends of aneurysm section holders 518, but also be circumferentially opposite of aneurysm section holders 518 from void 520. For example, referring to FIG. 15, aneurysm marker holders 1202 may be located near constraint terminal 1502, or as in other embodiments, located near constraint undulation 714, but may also be opposite aneurysm section holders 518 from void 520.

In addition to providing an indication of a vascular implant 500 location relative to an aneurysm, aneurysm markers 1204 may also allow an inference about a relative position between markers. For example, in an embodiment, markers may be differently shaped such that individual markers may be identified under imaging. In an embodiment, aneurysm marker holder 1202 located on first aneurysm section holder 602 may be an oval shape, while aneurysm marker holder 1202 located on second aneurysm section holder 604 may be circular or polygonal. Thus, while moving vascular implant 500 in vivo under fluoroscopy, it may be possible to determine the relative location of first aneurysm section holder 602 relative to second aneurysm section holder 604 by identifying the different marker shapes and inferring the relative positions therefrom.

In an embodiment, vascular implant 500 includes one or more end marker holders to hold respective aneurysm markers 1204. Referring again to FIG. 18, a side view of an end marker of a vascular implant is shown in accordance with an embodiment of the invention. One or more end marker 1802 may be located near vascular implant 500 ends within one or more end marker holder 1804. End marker holder 1804 may be integrally formed with a base ring 514 such as an end ring. For example, end marker holder 1804 may be laser cut along with base ring 514 and extend away from a base joint with a profile that encloses a marker area. Thus, in an embodiment, an end marker holder 1804 is incorporated at one or more base joint of an end ring. One or more end marker 1802 may be loaded into the end marker holders 1804, and thus, there may be a single end marker, or multiple end markers, at each end of vascular implant 500. End markers provide an indication of the ends of vascular implant 500 to facilitate accurate delivery and deployment of vascular implant 500. Thus, end markers 1802 may be located in any manner that facilitates accurate delivery and deployment of vascular implant 500. In an embodiment, end markers are triangularly shaped, however any marker shape may be used with sufficient marker material to provide visibility under a chosen imaging modality.

In an embodiment, vascular implant 500 includes a single end marker 1802 in each end ring. Furthermore, the two end markers 1802 may be axially aligned with each other and/or with any other feature of vascular implant 500. Accordingly, while viewing vascular implant 500 under fluoroscopy, an axis through the end markers may be used to infer a position of vascular implant 500. Thus, end markers may be viewed to allow a position of vascular implant 500 to be inferred for accurate positioning of vascular implant 500 during an interventional procedure. For example, delivery system may be advanced or rotated to accurately position vascular implant 500 relative to an anatomy of interest. Therefore, vascular implant 500 may be appropriately positioned prior to expanding base rings 514 against a vessel.

As described above, vascular implant 500 may have multiple marker holders 1804 to hold multiple markers 1802 according to design choice. Thus, markers may be located in any position, with any orientation and shape, according to design choice. For example, aneurysm markers 1204 and end markers may be shaped and formed to provide visibility under a chosen imaging modality. More specifically, visibility of aneurysm markers 1204 and end markers 1802 may be directly correlated with the size of the markers. Thus, the greater the volume and/or thickness of the markers, the more visible the markers may be under fluoroscopy. As a non-limiting example, aneurysm markers 1204 or end markers 1802 may have a thickness of about 0.0024-inch and a cross-sectional area in a range of about 0.00004 to 0.00005 square inch to provide visibility within most target anatomies. Thus, a marker volume may be about 10×10-8 to 12×10-8 cubic inches.

Aneurysm markers 1204 and end markers 1802 may be positioned and fixed within respective marker holders using various manufacturing processes, such as stamping, press fitting, adhesive or thermal welding. Alternatively or in combination with bonding processes, markers may be press fit within marker holders. For example, a slug of radiopaque material may be loaded into a marker holder and then stamped until it deforms into apposition with the marker holder. In an alternative embodiment, other processes may be used to load vascular implant 500 with a radiopaque material, such as coating, sputtering, or other known surface treatment processes.

Based on the description above, a vascular implant 500 may be provided that includes a plurality of base sections for anchoring vascular implant 500 in a vessel and an aneurysm section 517 between base sections. The aneurysm section 517 may be circumferentially discontinuous due to void 520 between aneurysm section holders 518. During expansion of vascular implant 500, void 520 may remain the same size, or it may increase to some degree. However, in either case, the aneurysm section 517 may extend substantially around the circumference in both the unexpanded state and the expanded state.

The description above relates primarily to various embodiments of structural features of vascular implant 500 for treating an aneurysm. These structural features are not necessarily specific to a particular implant materials. Thus, vascular implant 500 may be formed using a variety of materials. In an embodiment vascular implant 500 may be formed from materials that are suited to expansion using a balloon-expandable implant delivery system. For example, vascular implant 500 may be formed from stainless steel alloys, e.g., series 316L stainless steel, cobalt chrome alloys, e.g., L605 cobalt chrome or Elgiloy, MP35N, or platinum chrome, to name a few. Alternatively, vascular implant 500 may be formed from materials that are suited to self-expansion and delivery using a self-expandable implant delivery system. For example, vascular implant 500 may be formed from superelastic nickel titanium alloys. Alternatively, vascular implant 500 may be formed from plastically deformable polymers and self-expandable polymers, such as various formulations of polyurethane and polyethylene.

Vascular implant 500 may be fabricated using manufacturing processes that are known in the field of stent manufacturing. For example, balloon expandable or self-expandable vascular implants having a structure described in the embodiments above may be laser cut from raw material tubing. In an embodiment, raw superelastic nickel titanium alloy tubing with an outer diameter of 0.081-inch and a wall thickness of 0.004-inch may be used. Laser cutting may be followed by a combination of honing, cleaning, heat treating, micro blasting, cleaning, electro-polishing, and passivation processes. For example, in the case of balloon expandable implants, vascular implant 500 may be etched, passivated, and/or electropolished to achieve a surface finish that is clean, atraumatic to vessel tissue, and corrosion resistant. In the case of self-expandable implants, vascular implant 500 may be sand-blasted, etched, electropolished, and passivated to achieve a suitable surface finish.

In addition to finishing the surface of vascular implant 500, various steps may be followed to modify an expansion state of vascular implant 500. For example, various heat treatment steps may be applied to a self-expandable implant to provide a heat set material memory in the fully expanded configuration. Heat setting may involve expansion of base section and aneurysm section 517 to a desired configuration using a sequence of heat treating steps. For example, base section and aneurysm section 517 may be placed over a mandrel of a desired diameter in each step to sequentially increase the diameter to a deployment diameter, e.g., about 4 mm.

Vascular implant 500 may be loaded onto or into a delivery system in numerous manners. For example, in the case of a balloon-expandable implant, a crimping process may reduce the diameter of a laser cut vascular implant 500 to affix the implant struts to a non-compliant or semi-compliant balloon of a balloon delivery catheter. In the case of a self-expandable vascular implant 500, one or more crimping process may be applied to reduce the diameter of vascular implant 500 until it may be loaded into a delivery sheath of a self-expandable delivery system that constrains vascular implant 500 during delivery. For example, several crimping stages may be used to crimp vascular implant 500 to a cylindrical configuration in which base section and aneurysm section 517 is in a stacked state. As described above, constraint undulations 714 of vascular implant 500 may be substantially stiffer than other undulations in transition ring such that constraint undulations 714 prevent longitudinal aneurysm section holders 518 from overlapping during crimping and maintain aneurysm section holders 518 near each other during expansion.

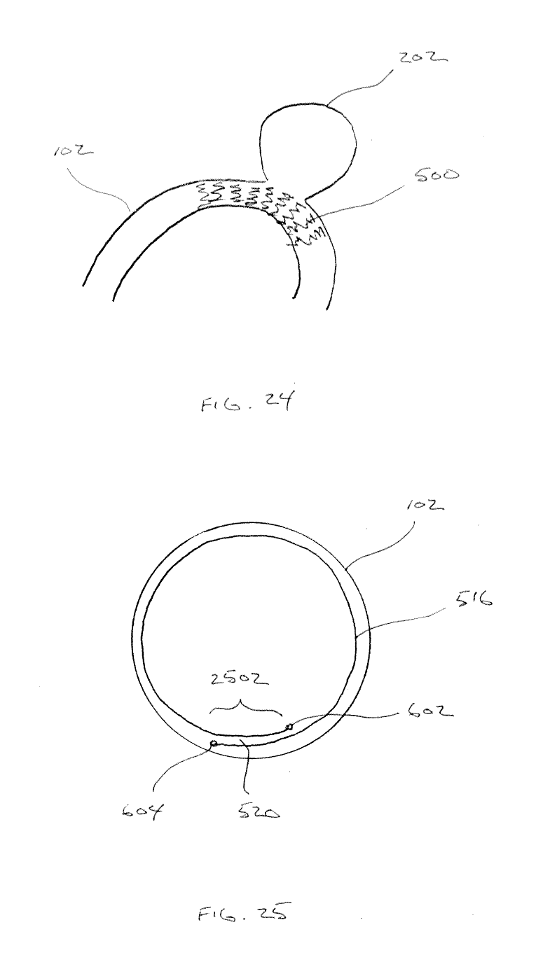

Referring to FIG. 24, a pictorial view of a vascular implant deployed at an aneurysm site is shown in accordance with an embodiment of the invention. Vascular implant 500 may be delivered to a portion of vessel having aneurysm using known delivery systems. For example, vascular implant 500 may be loaded onto or into a balloon expandable stent delivery system or a self-expandable stent delivery stent and tracked into alignment with aneurysm. More particularly, aneurysm markers 1204 and/or end markers 1802 of vascular implant 500 may be viewed under fluoroscopy to determine a position of aneurysm section 517 relative to aneurysm sac 202. Similarly, aneurysm markers 1204 and or end markers 1802 of vascular implant 500 may be viewed under fluoroscopy to determine a rotational orientation of void 520 relative to aneurysm sac 202. For example, the delivery system carrying vascular implant 500 may be advanced until aneurysm markers 1204 or end markers 1802 indicate that aneurysm section 517 is longitudinally aligned with an aneurysm gate, and rotated until aneurysm markers 1204 or end markers indicate that void 520 is circumferentially opposite of the aneurysm gate.

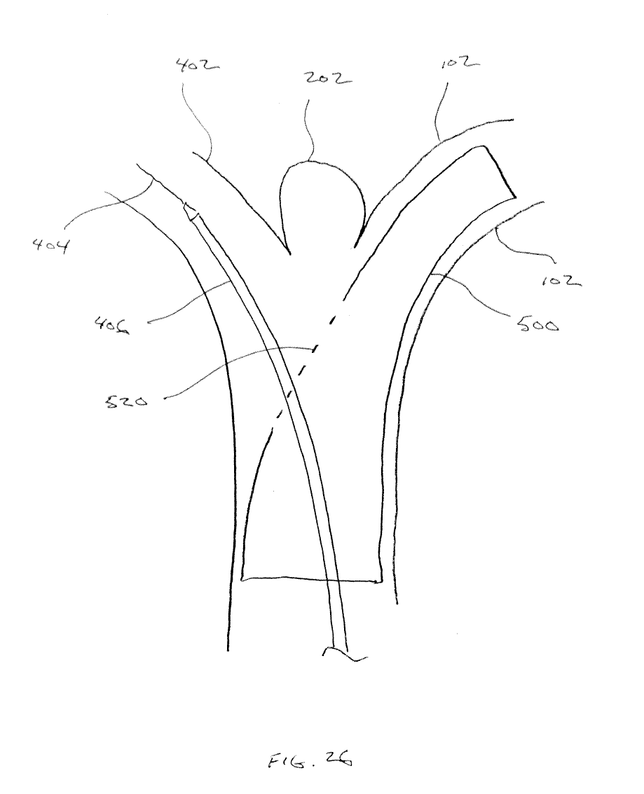

Referring to FIG. 25, a cross-sectional view of a vascular implant deployed at an aneurysm site is shown in accordance with an embodiment of the invention. After longitudinally and rotationally aligning vascular implant 500 relative to aneurysm sac 202 in a desired manner, vascular implant 500 may be deployed within vessel, e.g., by inflating a balloon element of a balloon expandable stent delivery system or retracting an outer sheath element of a self-expandable stent delivery system. Accordingly, vascular implant 500 may expand into contact with a wall of vessel 102.

In an embodiment, vessel may apply inward radial loading to vascular implant 500. For example, pulsatile loading in a wall of vessel may act upon vascular implant 500 to apply radial loading to aneurysm section 517. Furthermore, tortuosity near aneurysm site may result in loading on vascular implant 500 that promotes pancaking and kinking of typical stents. However, under similar circumstances, void 520 of vascular implant 500 may provide a stress relief that helps to prevent the pancaking effect. That is, in an embodiment, first aneurysm section holder 602 or second aneurysm section holder 604 may flex inward or outward relative to the other such that stresses are more evenly distributed through aneurysm section 517, and thus, aneurysm section 517 is not over-constrained. Thus, aneurysm section 517 may resist oval deformation of vascular implant 500. In an embodiment, when inward radial loading is applied by vessel, first aneurysm section holder 602 slides inward relative to second aneurysm section holder 604, causing aneurysm section 517 to overlap itself with void 520 between the overlapping portions of aneurysm section 517 in an overlap section 2502. Since aneurysm section 517 overlaps itself, an overall diameter of aneurysm section 517 may change to accommodate the changing diameter of vessel, such that vascular implant 500 remains cylindrical and conforms to appose vessel.

Referring to FIG. 26, a pictorial view of a delivery system passing through a vascular implant deployed at a site of a bifurcation aneurysm is shown in accordance with an embodiment of the invention. In an embodiment, vascular implant 500 may be axially and rotationally aligned relative to an ostium of a bifurcated aneurysm 202. More particularly, a delivery system carrying vascular implant 500 may be advanced under visualization, e.g., fluoroscopy, to align end marker 1802 and/or aneurysm markers 1204 such that void 520, which is represented as a dashed line portion of vascular implant 500, is aligned with branch vessel 402. Thus, guidewire 404 and catheter 406 may be delivered through void 520 from an inner lumen of vascular implant 500 into branch vessel 402. A second implant may be delivered from catheter 406 into branch vessel 402 to jail aneurysm 202 behind a pair of overlapping implants. In an embodiment, the secondary device may be another vascular implant 500 axially and rotationally positioned such that a respective void 520 is aligned with void of the second implant of the first vascular implant, and with main vessel. Therefore, after deploying the implants, balloon angioplasty catheters may be tracked through inner lumens of each vascular implant and expanded to perform a “touch up” or “kissing balloon” technique to cause vascular implants to appose both parent vessel 102 and branch vessel 402. In addition, a coil-containing micro catheter may be inserted through two voids 520 formed by two devices in order to unload and place coils inside the aneurysm sac to further accelerate clotting of the aneurysm. The combined gap 520 formed by two respective aneurysm sections of two devices may facilitate the insertion of the coil-containing micro catheter in order to advance and load coils into the aneurysm sac. Aneurysm 202 at the bifurcation ostium may therefore be properly jailed to allow sufficient blood flow through patient vasculature.

These and other processes may be performed in accordance with the skill in the art. For example, coating processes may be used to coat one or more surface of vascular implant 500 with therapeutic agents, including drugs that have been used in the field of drug-eluting stents, e.g., paclitaxel, zotarolimus, everolimus, sirolimus, etc. These agents may be used alone or in combination with polymer carriers, such as biostable or biodegradable polymers that may be loaded to retain and time-release a therapeutic agent. Thus, the manufacturing processes provided above are illustrative and not limiting of the range of manufacturing processes that may be used to form vascular implant 500 and to prepare the implant for delivery to an aneurysm location within a patient.

In the foregoing specification, the invention has been described with reference to specific exemplary embodiments thereof. It will be evident that various modifications may be made thereto without departing from the broader spirit and scope of the invention as set forth in the following claims. For example, although the description above may refer to a location of a specific ring or arc scaffold structure or sub-element, e.g., proximal transition ring or distal transition ring, the scaffold structures and sub-elements of vascular implant may be combined and located in any manner to form vascular implant having the features described above. The specification and drawings are, accordingly, to be regarded in an illustrative sense rather than a restrictive sense.

What is claimed is:

1. A vascular implant having an unexpanded state and an expanded state, the vascular implant comprising:
a proximal transition ring and a distal transition ring, the proximal transition ring having a plurality of ring undulations contiguous about a longitudinal axis such that the proximal transition ring is continuous around a circumference of the vascular implant;
a plurality of aneurysm section holders extending longitudinally from respective ring undulations to the distal transition ring, the plurality of aneurysm section holders separated by a void; and
an aneurysm arc having a plurality of arc undulations contiguous about the longitudinal axis and opposite of the plurality of aneurysm section holders from the void such that the aneurysm arc is discontinuous and extends substantially around the circumference in the unexpanded state and the expanded state.

2. The vascular implant of claim 1, wherein the ring undulations are contiguously coupled with each other by a plurality of transition joints, wherein the arc undulations are contiguously coupled with at least one of each other or an aneurysm section holder by a plurality of arc joints, and wherein a plurality of transition connectors couple the plurality of transition joints with the plurality of arc joints.

3. The vascular implant of claim 2, wherein the vascular implant includes a same number of transition joints in the proximal transition ring as arc joints in the aneurysm arc.

4. The vascular implant of claim 1, wherein the plurality of ring undulations includes a constraint undulation and a plurality of transition undulations, and wherein the plurality of aneurysm section holders extend longitudinally from the constraint undulation to the distal transition ring.

5. The vascular implant of claim 4, wherein the constraint undulation includes a first composite stiffness, wherein each of the plurality of transition undulations include a respective second composite stiffness, and wherein the first composite stiffness is greater than each of the respective second composite stiffness.

6. The vascular implant of claim 5, wherein the constraint undulation includes a plurality of constraint struts having a first length and the plurality of expansion undulations include a plurality of expansion struts having a second length, and wherein the first length is shorter than the second length.

7. The vascular implant of claim 5, wherein the constraint undulation includes a constraint joint having a first width, wherein the plurality of expansion undulations include a plurality of transition joints having a second width, and wherein the first width is greater than the second width.

8. The vascular implant of claim 4, wherein the proximal transition ring includes a stack undulation, wherein the stack undulation includes a plurality of stack struts interconnected by a stack joint, and wherein the stack struts include respective ends coupled with respective constraint struts of the constraint undulation.

9. The vascular implant of claim 1, wherein the void includes a width along a longitudinal length of the arc segment, and wherein the width has a distance less than about ten percent of the circumference of the vascular implant in the unexpanded state and the expanded state.

10. The vascular implant of claim 9, wherein the aneurysm section holders extend substantially straightly in an axial direction from the respective ring undulations to the distal transition ring.

11. The vascular implant of claim 9, wherein the void includes a port between the proximal transition ring and the distal transition ring, and wherein the port includes a shape configured to allow a catheter to pass through the void from an inner lumen of the vascular implant.

12. The vascular implant of claim 11, wherein the shape includes a projected area selected from a group consisting of an ellipse and a polygon.

13. The vascular implant of claim 9, wherein a geometric surface defined by the void is configured to remain substantially the same when the vascular implant expands from the unexpanded state to the expanded state.

14. The vascular implant of claim 9, wherein a geometric surface defined by the void is configured to increase when the vascular implant expands from the unexpanded state to the expanded state.

15. The vascular implant of claim 1 further comprising:
one or more aneurysm marker holder adjacent to the plurality of aneurysm section holders; and
an aneurysm marker in each aneurysm marker holder.

16. The vascular implant of claim 15, wherein the one or more aneurysm marker holder is circumferentially between at least one of the plurality of aneurysm section holders or a plurality of constraint struts of a constraint undulation of the proximal transition ring.

17. The vascular implant of claim 1 further comprising:
a first end marker holder opposite of the proximal transition ring from the aneurysm arc;
a second end marker holder opposite of the distal transition ring from the aneurysm arc; and
an end marker in each end marker holder.

18. The vascular implant of claim 1 further comprising a radial connector interconnecting the plurality of aneurysm section holders at an intermediate location between the proximal transition ring and the distal transition ring.

19. The vascular implant of claim 18, wherein the radial connector includes an intermediate aneurysm marker holder and an intermediate aneurysm marker in the intermediate aneurysm marker holder.

20. The vascular implant of claim 1, wherein the aneurysm arc includes a first arc pattern circumferentially between a first of the plurality of aneurysm section holders and a plane, and a second arc pattern circumferentially between a second of the plurality of aneurysm section holders and the plane, wherein the plane passes through the void and the longitudinal axis, and wherein the first arc pattern and the second arc pattern are symmetric.

21. The vascular implant of claim 15, wherein the one or more aneurysm marker holder includes a first aneurysm marker holder adjacent to a first aneurysm section holder and a second aneurysm marker holder adjacent to a second aneurysm section holder, and wherein a first aneurysm marker in the first aneurysm marker holder is shaped differently than a second aneurysm marker in the second aneurysm marker holder.
